(19) United States
(12) Patent Application Publication
(10) Pub. No.: US 2007/0221225 A1
(43) Pub. Date: Sep. 27, 2007

(54) Simulated Altitude Method and Apparatus

(76) Inventors: Larry Kutt, Boulder, CO (US); Shaun Wallace, Cardiff, CA (US); William Reid, Longmont, CO (US); Bennett Scharf, Boulder, CO (US); Frank L. Caruso, Golden, CO (US); Tom Damian, Boulder, CO (US); Doug Ogden, Lyons, CO (US)

Correspondence Address: SHERIDAN ROSS PC, 1560 BROADWAY, SUITE 1200, DENVER, CO, 80202, US

(21) Appl. No.: 11/626,770

(22) Filed: Jan. 24, 2007

Related U.S. Application Data
(60) Provisional application No. 60/761,995, filed on Jan. 24, 2006.

(51) Int. Cl. A61G 10/00; A61M 16/00; A62B 31/00

(52) U.S. Cl. 128/205.26; 128/202.12; 128/204.23

(57) ABSTRACT

An altitude simulation system is disclosed for simulating an altitude within an enclosure or mask, wherein various improvements are provided, including: (a) a more effective use hypoxpic air generated by the system via recirculating techniques and improvements in air leakage, (b) improvements in determining when a simulated altitude is reached, (c) improvements in controlling hypoxic air generators so that peak electrical power loads are reduced and there are enhanced failsafe features for protecting the health of users, (d) using a pulse oximetry device for measuring oxygen saturation in a user's blood to thereby vary the oxygen content in the air provided to the user.

### CROSS-REFERENCE TO RELATED APPLICATIONS

The present application claims the benefit of U.S. Provisional Patent Application No. 60/761,995, filed Jan. 24, 2006, which is fully incorporated herein by reference.

### BACKGROUND

When a mammal is exposed to hypoxia a series of adaptive accommodations occur. Many of these adaptations are beneficial to the mammal. Reduced oxygen levels associated with altitude produce a variety of beneficial effects and physiological accommodations. However, altitude is not available to the majority of the population, as a result there is a great deal of interest in altitude simulation.

Normabaric hypoxic altitude simulation is used for, but not limited to, athletic training, weight loss, hypoxic preconditioning, and the treatment of certain medical conditions including hypoplastic left heart syndrome.

Hypoplastic left heart syndrome is a congenital defect that affects newborn babies. Hypoxia is often used to produce physiological effects that keep the baby alive while waiting for surgical treatment or heart transplant. Present methods for safely providing such an hypoxic environment for babies are expensive, cumbersome, difficult to regulate, and in some cases, pose certain risks to the babies.

Research shows that when cells are exposed to hypoxia, they respond better to future and more severe exposures to hypoxia. Hypoxic preconditioning (HPC) with mild non damaging hypoxia confers a kind of immunity to damage from severe hypoxia. This is useful to prepare patients for surgery and to prevent damage from ischemia such as may occur after a stroke or heart attack.

Additionally, recent studies show that erythropoietin, EPO, can be used to treat nerve damage in the brain and spinal cord. This damage may be caused by, e.g., a stroke. Research studies suggests that EPO helps protect healthy neurons from being damaged from existing damaged neurons. Since EPO is naturally produced by living bodies as a response to acclimatization to high altitude, hypoxia induced, via an altitude simulator, can be used as a treatment for this and other conditions.

Since the percentage of oxygen remains constant (20.9%) in ambient air regardless of elevation, a decrease in ambient oxygen is a result in the reduction of the partial pressure of oxygen that occurs as altitude increases. Such a reduction in oxygen partial pressure can be simulated in an enclosure regardless of the attitude of the enclosure for producing an hypoxic atmosphere therein. Altitude simulation systems can simulate changes in attitude in many ways; the following are just a few examples: simulation by changing partial pressure in a hyperbaric chamber, simulation by decreasing the percentage of oxygen relative to ambient air, or by increasing the nitrogen content of normobaric air. However, heretofore such altitude simulation systems have been inefficient, have reduced safety features, inaccurate in attaining/maintaining a desired simulated altitude, and expensive. Accordingly, it is desirable to have a enhanced altitude simulation system that addresses such problems with previous altitude simulation systems.

To provide further background regarding the health and performance benefits of hypoxic environments and a additional general background for altitude simulation systems, the reference “A Practical Approach to Altitude Training” by Dr. Ed Burke is provided in Appendix E herein.

Additionally, each of the following U.S. patents are fully incorporated by reference herein:
U.S. Pat. No. 5,188,099 issued February 1993 to Todeschini et al;
U.S. Pat. No. 5,207,623 issued May 1993 to Tkatchouk et al.;
U.S. Pat. No. 5,383,448 issued January 1995 to Tkatchouk et al.;
U.S. Pat. No. 5,467,764 issued Nov. 21, 1995 to Gamow;
U.S. Pat. No. 5,860,857 issued January 1999 to Wasastjerna et al.;
U.S. Pat. No. 5,887,439 issued March 1999 to Kotliar;
U.S. Pat. No. 5,924,419 issued July 1999 to Kotliar;
U.S. Pat. No. 5,964,222 issued October 1999 to Kotliar;
U.S. Pat. No. 6,009,870 issued January 2000 to Tkatchouk;
U.S. Pat. No. 6,314,754 issued November 2001 to Kotliar;
U.S. Pat. No. 6,334,315 issued January 2002 to Kotliar;
U.S. Pat. No. 6,401,487 issued June 2002 to Kotliar;
U.S. Pat. No. 6,418,752 issued July 2002 to Kotliar;
U.S. Pat. No. 6,502,421 issued January 2003 to Kotliar;
U.S. Pat. No. 6,508,850 issued January 2003 to Kotliar;
U.S. Pat. No. 6,557,374 issued May 2003 to Kotliar;
U.S. Pat. No. 6,560,991 issued May 2003 to Kotliar;
U.S. Pat. No. 6,565,624 issued May 2003 to Kutt, et al.;
U.S. Pat. No. 6,827,760 issued December 2004 to Kutt, et al.;
U.S. Patent Application Publication No. 2001/0029750 issued October 2001 to Kotliar;
U.S. Patent Application Publication No. 2002/0016343 issued February 2002 to Crocker et al.;
U.S. Patent Application Publication No. 2002/0023762 issued February 2002 to Kotliar;
U.S. Patent Application Publication No. 2002/0083736 issued July 2002 to Kotliar;
U.S. Patent Application Publication No. 2002/0088250 issued July 2002 to Kotliar; and
U.S. Patent Application Publication No. 2003/0074917 issued April 2003 to Kotliar.

### Description of Terms

Ambient—An environment that surrounds a specified object or space but does not include the space within the object. More particularly, a gaseous environment that occurs surrounding the object (e.g., surrounding a room, tent, etc).
Normabaric—Refers to the nominal barometric pressure in an ambient environment. In particular, this is the atmospheric pressure at the installation location.
Nominal Amount of Oxygen—The normal percentage of oxygen available in a fluid, for the earths atmosphere this percentage is typically 20.9%.
Hypoxic—Oxygenated fluid with less than the nominal amount of oxygen
Hyperoxic—Oxygenated fluid with more than the nominal amount of oxygen
Normoxic—Oxygenated fluid with the nominal amount of oxygen.
Enclosure—A space for containing a gaseous environment such as a hypoxic environment during an active altitude simulation; also referred to as the following herein:
   environmental enclosure,
   room,
   tent, and/or
   environmental chamber.
Hypoxic air generator (HAG)—A device that separates normoxic air into oxygen and hypoxic air; also referred to as the following herein:
   Hypoxic/Normoxic Air Generator,
   air unit,
   oxygen concentrator, and/or
   concentrator.
Controller—A device that controls air generators and other various components necessary to an altitude simulation system, wherein the air generators may generate at least one of hypoxic and/or hyperoxic air; also referred to as the following herein:
   control box, and/or
   system controller.
Oxygen valve system—An electronically controlled valve system whose actuation determines the type of air flow from a HAG to, e.g., an enclosure.
Signal—An electrical signal emanating from a control device for controlling and/or communicating with another electrical device.
Control Link—An electrical conductor that carries signals from a control device to a device(s) being controlled; also referred to as the following herein:
   control cable, and
   data link.
Channel—A device/HAG or a series of devices/HAGs together with cabling for receiving signals from a controller, and air lines for supplying an air mixture to an enclosure or mask.
Blow-through—A process of supplying a quantity of hypoxic or nomoxic air to an altitude simulation system wherein an enclosure for the system is fed a continuous supply of the hypoxic or nomoxic air while in use and the enclosure allows the excess air to dissipate into the ambient environment; also referred to as the following herein:
   open loop.
Closed Loop—A process of supplying a quantity of hypoxic or nomoxic air to an altitude simulation system wherein an enclosure for the system is fed a supply of hypoxic or nomoxic air that is fully or partially recirculated from the enclosure while in use.
Feed line—Tubing that connects a hypoxic/hyperoxic generator to an enclosure and supplies the enclosure with generated air.
Recirculation Line—Tubing that connects an enclosure to a hypoxic/hyperoxic generator intake to allow recirculation of air to the enclosure.
Molecular sieve beds—A filter for filtering oxygen from air, wherein operation is by forcing pressurized air through crystalline zeolite which captures the oxygen molecules from an air intake and releases the captured oxygen through a predetermined port, and hypoxic air through another predetermined port.
High Altitude Refinement—A standard two-point transducer calibration process that is used at the lower and upper bounds of the intended operating range of the transducer; also referred to as the following herein:
   upper limit calibration
Set point—A desired simulated altitude within an enclosure/mask, or an oxygen saturation in the blood of a user; a set point may be in terms of an O₂ percentage, a simulated altitude (e.g., in feet above sea level), or an arterial oxygen saturation.

### SUMMARY

The present disclosure describes a means of controlling hypoxic air and normoxic air from a hypoxic/normoxic air unit(s) to modulate hypoxia and to control air quality supplied to a user when simulating an atmospheric environment corresponding to a desired altitude to thereby initiate a physiological response. In particular, a method and apparatus for simulating altitude within an enclosure are disclosed. More particularly, various enhancements to an altitude simulation system are disclosed for making such systems more efficient, safer, more accurate, and/or better adapted to user needs. It is within the scope of the present disclosure that any combination of components and processes for these enhancements may be provided in an embodiment of an altitude simulation apparatus and method disclosed herein. Thus, additional novel features disclosed herein are such combinations.

The invention includes a method and system for simulating altitude changes in an enclosed space or a breathing mask, and is particularly directed to a method and system in which controlled oxygen and carbon dioxide levels are monitored and adjusted to provide desired physiological benefits derived from a person or animal spending time in an altitude environment as a treatment to medical conditions. High and low oxygen environments affect the physiology in different ways providing health and athletic benefits. In particular, a normabaric hypoxic altitude simulation is disclosed which is used for, but not limited to, athletic training, weight loss, the treatment of diabetes, hypoxic preconditioning (HP), and the treatment of certain medical conditions including hypoplastic left heart syndrome and/or rehabilitation of heart attack patients. Accordingly, various techniques and components are disclosed as a means of controlling hypoxic air and normoxic air from a hypoxic/normoxic air generator(s) to modulate hypoxia and to control air quality.

In one embodiment, the invention includes an Air Intake Module that is placed inline ahead of the intakes for the air generating units. The Air Intake Module regulates the source of the intake air from the re-circulated air from within the hypoxic enclosure and the fresh air from the ambient via an electronically controlled flapper valve and actuator.

The Air Intake Module of the present disclosure serves as a three-way valve to switch between fresh air and re-circulated intake air for an hypoxic air generator; the housing for the Air Intake Module accommodates an air filter, and includes a manifold to distribute flow to multiple hypoxic air generators. The actuated three-way valve allows automated switching between recirculation and fresh-air intake modes, wherein recirculation refers to a volume of air in the simulated altitude enclosure (labeled as “1” in FIG. 1) that is used as the intake air for the hypoxic air generator(s). Recirculation of enclosure air allows faster ramp-up to a set-point simulated altitude. Subsequent switching to at least a partially non-recirculation mode once the simulated altitude set-point is achieved allows for greater occupant comfort by removing waste gasses such as CO₂.

At start-up with an enclosure at a normal ambient oxygen level, a controller activates the hypoxic air generators and signals an actuator motor of the Air Intake Module to move the valve to the recirculation position (blocking a fresh-air intake port). Once the simulated altitude set-point is achieved the controller signals the actuator motor to move the valve to the fresh-air intake position (close the recirculation port) and fresh air is processed through the hypoxic air generators.

All currently-available prior art three-way valves are over-designed (and thus over-priced) for this application, which is low-pressure with a benign (non-corrosive) fluid (e.g., air). Such prior art valves are designed for high pressures, corrosive chemicals, vacuums, etc., and require an expensive servo motor for actuation.

The purpose of the filter provided in the Air Intake Module is to protect the compressor from premature wear due to particulate sizes greater than 10μ (microns) in the intake air. Including the manifold function in the Air Intake Module assembly reduces flow losses due to multiple in-line connectors as well as reducing the number of components.

Many obese patients suffer from obstructive sleep apnea (OSA) or other apneas. As such they require the use of a CPAP machine to assist breathing during sleep. Hypoxic altitude simulation systems have been shown to induce weight loss. To facilitate the use of hypoxic altitude simulation systems in obese patients at least one embodiment of the invention includes a method and apparatus for combining a CPAP machine with an hypoxic altitude simulation system.

In one embodiment, the present disclosure includes a manifold that, when seals to an gas separation filter, recaptures hypoxic gases from the gas separation filter and ducts the recaptured hypoxic air through a desired exhaust port. This results in less loss of hypoxic gas than in prior art systems, and allows for an increase of air flow of hypoxic gas to a user (human or mammal) of 10-25%.

In one embodiment, the invention includes two components that are used in conjunction with a simulation environmental enclosure, hypoxic air generator(s), and the necessary attachments. The first component of the invention includes a controller with sensors and processors that monitor and control the airflow and air type (hypoxic or normoxic) to the hypoxic environmental enclosure in which the controller may reside. The second includes an oxygen valve system that controls the type of air flowing through the hypoxic air generators via control signals from the controller.

In at least one embodiment, the invention includes a multi-channel altitude simulation system that has the capability of turning on boost channels when more hypoxic air may be required while turning off boost channels when less hypoxic air is required and provides for quieter, cooler, and more economical operation.

In at least one embodiment, the invention includes a method and apparatus for controlling oxygen levels in individuals based on their specific physiology of by means of measuring the oxygen saturation in the blood and by monitoring and adjusting oxygen levels in a controlled environment. Prior art systems for regulating the oxygen levels in air did not account for individual differences in response to different oxygen levels. The present embodiment directly measures a user's response to hypoxia or hyperoxia by means of a pulse oximeter, wherein the signals therefrom are used as input to a controller that controls hypoxic air generators capable of changing the oxygen content of air provided to the user.

Additional features and benefits of the present disclosure are provided in the description hereinbelow, and accompanying drawings.

### BRIEF DESCRIPTION OF THE DRAWINGS

FIGS. 1 and 2 show how an Air Intake Module (AIM) 5 is integrated into an altitude simulation system. In particular, these figures show a hypoxic altitude simulation system (HASS) with multiple air generating units (3), together with the novel Air Intake Module 5 for controlling the composition of the air input to the air generating units.

FIG. 3 shows an exterior view of an embodiment of the Air Intake Module 5.

FIG. 4 shows an interior view of the Air Intake Module 5.

FIG. 5 shows additional interior views of the Air Intake Module 5.

FIGS. 6 and 7 show additional exterior views of the Air Intake Module 5.

FIG. 8 shows another interior view of the Air Intake Module 5.

FIG. 9 depicts a Continuous Positive Airway Pressure (CPAP) 102 device being used in a controlled hypoxic environment where the input air to the CPAP may be hypoxic via the air within the enclosure.

FIG. 10 depicts a different embodiment where the input to the CPAP device 102 comes directly from a hypoxic air generator without the need for an enclosure.

FIG. 11 depicts a typical interface between a hypoxic air generator 3 and a custom exhaust manifold 207 where an o-ring 209 seals to the housing 210 of the generator 3.

FIG. 12 depicts one preferred interface where the manifold 207 seals to the face of the hypoxic air generator 3 with an o-ring 209.

FIG. 13 depicts one typical setup of an altitude simulation system with a single air unit control channel 307, and multiple hypoxic air generating units 3 (e.g., 3a through 3d) connected to the channel 307.

FIG. 14 depicts the oxygen valve system 319 that may be used by a controller 2 for an altitude simulation system to switch between hypoxic and normoxic air.

FIG. 15 is a schematic of the internal components of a controller 2 for controlling an altitude simulation system.

FIG. 16 is a picture of the internal components of a controller 2 for controlling an altitude simulation system.

FIG. 17 shows three hypoxic air generators 3a, 3b, and 3c daisy chained together.

FIG. 18 is another picture of the internal components of a controller 2 for controlling an altitude simulation system.

FIG. 19 depicts an altitude simulation system that employs three channels 307a through 307b with single air units supplying air for each channel. The three channels consist of a main and two boost channels: A and B. A boost channel is a combination of air generating units (3), control links 307, and feed line (10) into the enclosure (5).

FIG. 20 is a schematic of a multi-channel multi-unit altitude simulation system.

FIG. 21 shows a pulse oximeter 504 for signaling an altitude simulation controller 2 to adjust oxygen levels in an enclosure 1 via a hypoxic air generator 3 and thereby reach the desired oxygen concentration in a user's body.

FIG. 22 show an additional embodiment of a pulse oximeter 504 for use in an enclosure 1 for simulating altitude.

FIG. 23 shows an embodiment of a ventilation subsystem 600 for ventilating an enclosure 1.

### DETAILED DESCRIPTION

1. Air Intake Module for Hypoxic Altitude Simulation: A First Aspect of the Present Disclosure.

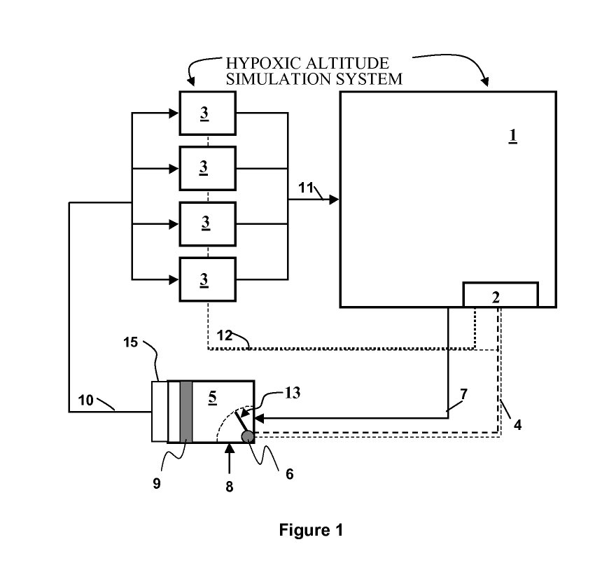

The present section discloses an Air Intake Module (AIM) 5 (shown in FIGS. 1 through 8) designed to be integrated into an electronically controlled multi-hypoxic air generator system to allow for recirculation of hypoxic air into the intake system to bring the environment to simulated altitude quicker and/or with less hypoxic air generating units.

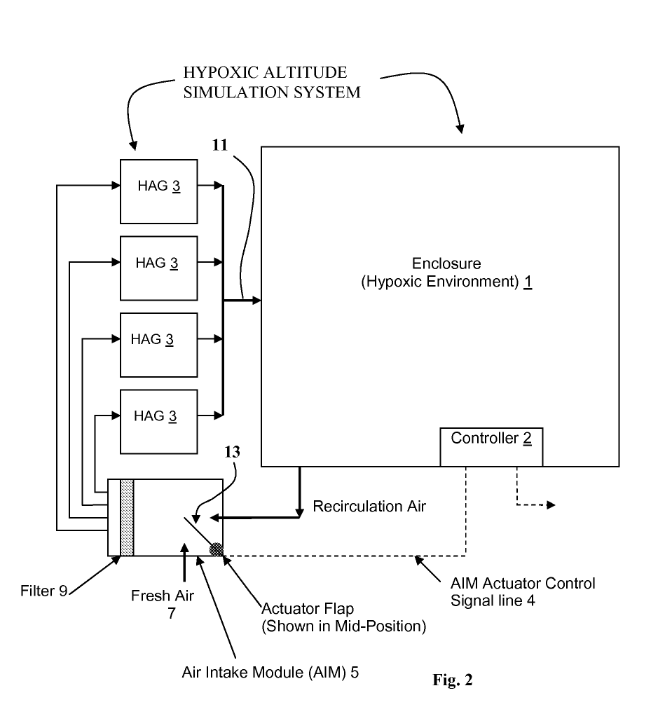

The Air Intake Module 5 includes as a three-way valve assembly 6 (e.g., FIG. 4) to switch between fresh air from a fresh air intake 8 and re-circulated air from a re-circulated air intake 7 for supplying air to the hypoxic air generators 3 (FIGS. 1 and 2). A housing 24 for the AIM 5 includes therein an air filter support 28 for retaining an air filter 9 (FIGS. 1 and 2) therein. The AIM 5 also includes a manifold 15 to distribute outbound air flow to multiple hypoxic air generators 3. In the embodiment of FIG. 1, the multiple outbound air flows are represented by a single line labeled 10 representing the generator air intake lines.

The actuated three-way valve assembly 6 allows automated switching between at least recirculation and fresh-air intake modes, wherein the term recirculation refers to a volume of air in the enclosure 1 (FIGS. 1 and 2) that is used as the intake air for the hypoxic air generator(s) 3. Recirculation of air from enclosure 1 allows faster ramp-up to a desired set-point for a desired simulated altitude. Thus, the AIM 5 is able to switch between a flow-through mode (where air is re-circulated), and a fresh air intake mode (where the input air to the air generators 3 is not re-circulated, but instead external ambient air is input to the generators 3). Accordingly, by switching to the fresh air intake mode once the simulated altitude set-point is achieved, the resulting ambient air intake in combination with purposefully designed air leakage in the system and the enclosure 1, allow CO₂ and other gases to appropriately vent to the ambient environment. Such an increase in ambient air allows for greater enclosure 1 occupant comfort by more effectively removing waste gasses such as CO₂. Additionally, switching to flow-through mode also allows for any given number of air units (e.g., hypoxic air generators 3) to be used to create a higher simulated altitude inside the enclosure 1, than if the intake air was external ambient air. Also, by switching to flow-through mode, the enclosure 1 can accept a higher natural infiltration rate of fresh air (leakage) into the room, yet still create the desired simulated altitude. Note that by controlling the switching between the two modes, the simulated altitude in the enclosure 1 can be better controlled for maintaining the desired air environment in the enclosure.

Referring to FIG. 1 in more detail, a controller module 2 located within the hypoxic enclosure 1 monitors various environmental parameters of the enclosure 1. The controller 2 generates output signals 12, 4 that control the operation of the hypoxic air generators 3 and the valve position of the three-way valve assembly 6. The valve position determines whether the hypoxic air generator intake lines 10 provide air from the re-circulation air line 7 or the fresh air intake 8 from the ambient environment external to the enclosure 1. There is a filter 9 located in the Air Intake Module 5 to prevent particles greater than a predetermined size from entering the system and causing premature component wear (e.g., to the generators 3). An additional benefit of the Air Intake Module 5 is that in the flow-through mode, it allows the hypoxic air generators 3 to remove more oxygen from the already partially de-oxygenated air.

At start-up of the hypoxic altitude simulation system wherein the enclosure 1 contains a normal oxygen level, the controller 2 activates the hypoxic air generators 3 and signals the actuator motor (not shown) for the three-way valve assembly 6 (also referred to as a flapper valve, as one of ordinary skill in the art will understand) to move a flapper 13 (FIGS. 1, 2, 4) to the recirculation position (blocking the fresh-air intake port). Note that in the embodiment of FIG. 4 the flapper includes a solid air impermeable planar flap 14 having a block of a foam or sponge material 16 on each side of the flap, wherein the material 16 substantially reduces the noise when the flapper 13 pivots about the actuator shaft 17 according to the bi-directional arrow 19 for closing the air line 7 or the air intake 8. The material 15 need not be air impermeable, but should at least restrict the any air infiltration, of a corresponding air input from one of the air line 7 or the air intake 8 closed by the flapper 13, to less than 10% of the volume that would otherwise flow through when this corresponding input open.

Once the enclosure 1 reaches a programmed set point for the desired oxygen content in the enclosure, the controller 2 switches the intake supply air from the re-circulation air line 7 to receiving air from the ambient air intake 8 via a signal on the control line 4 which actuates the flapper valve motor (not shown) within the Air Intake Module 5. In either case, the supply air 10 for the air generators 3 comes from the Air Intake Module 5 and enters the enclosure 1 from the hypoxic air generators 3 through the hypoxic air line 11 (FIGS. 1 and 2).

Currently available prior art three-way valves are over-designed (and thus over-priced) for controlling air flow for the hypoxic altitude simulation system (HASS) since the air flow for this system is low-pressure with a benign (non-corrosive) fluid (i.e., air). In particular, the air pressure within the Air Intake Module 5 is in the range of 0 to 30 PSI. The prior art valves are designed for high pressures, corrosive chemicals, vacuums, etc., and require an expensive servo motor for actuation.

The purpose of the filter 9 is to protect the compressor (not shown) within each of the air generators 3 from premature wear due to particulate sizes greater than 10μ in the intake air to the compressor. Additionally, note that a manifold 15 in the Air Intake Module 5 reduces flow losses due to multiple in-line connectors (shown in, e.g., FIG. 4, the three connectors are collectively identified by the label “15”) as well as reducing the number of components for the HASS.

In at least one embodiment of the present invention a means for reducing the attenuation of sound (primarily compressor/pump noise) through the Air Intake Module 5 may be incorporated into the present invention for controlling hypoxic air and normoxic air. The means for reducing such sound includes, but is not limited to, sound reduction materials and/or a tortuous air-flow path via baffles or other means as one of ordinary skill in the art will understand.

Another embodiment of the HASS allows control of the valve 6 position to a mid-point (i.e., “throttling”) valve position to provide a mix of re-circulated and ambient air supply to the hypoxic air generators. Such a mixture from both air intakes 7 and 8 may be used in the event that the flapper 13 is repeatedly being switched between closing intake 7 and intake 8 within a short intervals of time, e.g., every 30 seconds to 2 minutes. Additionally, the midpoint position can provide greater air flowing through the AIM 5 in the event that the HASS is not responding to a set point.

The Air Intake Module 5 may include a safety feature as well. In particular, the flapper 13 may have a default position (should the power be cut) is that will cause the flapper to move to the position blocking any recirculation of air (e.g., via a strong return spring for biasing the flapper to close the intake 8). Without this feature it may possible for the enclosure 1 to reach dangerously high simulated altitudes.

2. Use of Hypoxic Air as Input to a CPAP Device: A Second Aspect of the Present Disclosure.

Continuous Positive Airway Pressure (CPAP) is commonly used as a treatment for Obstructive Sleep Apnea (OSA). By pressurizing the ambient air and delivering it to a patent's airway via a mask or special pillow, the positive pressure keeps the users airways open during sleep.

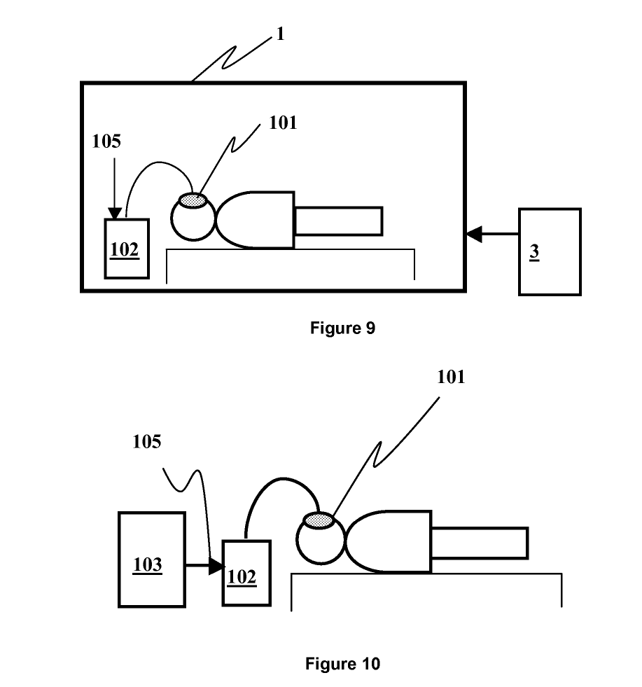

In FIG. 9, the CPAP device 102 supplies pressurized hypoxic air to the user via a mask or special pillow 101. The enclosure 1, which contains the user, is supplied hypoxic air via a hypoxic air generator 3. Since the CPAP resides inside the enclosure 1, the air it compresses to make positive pressure airflow is already hypoxic (as indicated as the arrow 105) and therefore the positive air supplied to the user is also hypoxic even though it is pressurized.

In FIG. 10, the hypoxic air supplied 105 to the CPAP 102 comes directly from a hypoxic air generator 3. The hypoxic air is pressurized by the CPAP 102 and is delivered to the user via a mask or special pillow. This alternative embodiment does not require the use of an enclosure.

3. Custom Air Unit Exhaust Manifold: A Third Aspect of the Present Disclosure.

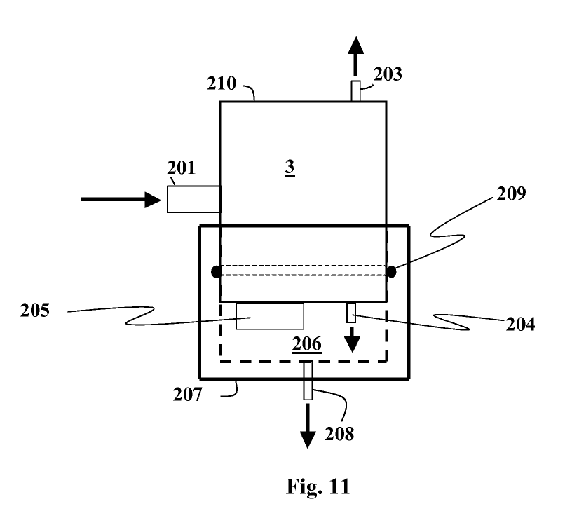

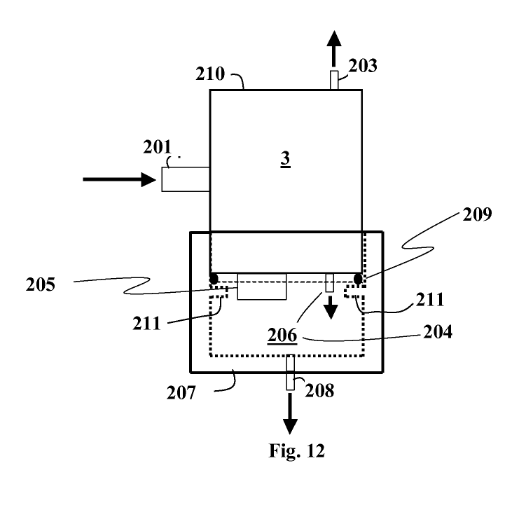

FIGS. 11 and 12 show embodiments of an exhaust manifold 207 that may be used to capture gas exhausted from an air separation filter 3 (e.g., an oxygen or nitrogen separator) via exhaust port 204, and then duct this captured exhaust gas through an exhaust port 208. Some common types of air separation processes are oxygen and nitrogen separation. Through these processes, nitrogen, oxygen, hypoxic air, and hyperoxic air can be produced.

More specifically, FIGS. 11 and 12 depict an air separation filter 3 and an air exhaust manifold 207 that is sealed to the air separation filter so that at least 99% of the air within the manifold 207 can only exit through the exhaust port 204.

A typical air separation filter 3 has 3 ports: an intake port 201, and two exhaust ports 203, and 204. The gas separation process provided by the filter 3 intakes a mixed gas, usually ambient air (or variation thereof), and the process separates one gaseous compound G₁ (e.g., oxygen) from the intake gas, and exhausts G₁ to one port (e.g., port 203), and exhausts the remainder of the intake gas through the other port (e.g., port 204).

Some gas separation filters are designed to allow the gasses to flow directly through the filter, wherein there is a single inlet with two exhaust streams. Such a filter does not incorporate moving parts and the gas flow is continuous in a single direction through the filter. However, other separation filters incorporate a valving system that allows the gas to flow in multiple directions through the filter and utilize pressure changes in the filter to facilitate separation of gasses. It is preferred that the separation filters 3 are mechanically operated. Moreover, such filters 3 may have exposed mechanical components 205 (e.g., a motor and/or a transmission, not individually shown in the figures) on the outside of the separation filter 3. Applicants have discovered that such exposed mechanical components 205 provide pathways for substantial leakage of the exhaust gas that would otherwise be exhausted through port 204. In particular, although a majority of the gas is intended to exit port 204, up to 25% of this gas may leak through the mechanical components 205. Such leakage is believed to be substantially due to the exhaust gas G₁ in the filter 3 being pressurized (e.g., 0 to 165 psi, more particularly, 0 to 35 psi). Accordingly, this gas will travel the path of least resistance and vent into the environment external to these components.

The custom manifold 207 fits over, e.g., an end of the separation filter 3 for encapsulating the mechanical components 205 and the exhaust port 204. The manifold 207 can be made of plastic or metal. Pressure formed or injection molded plastic may be preferred for the manifold 207. Other options include vacuum formed plastic or cast aluminum. The manifold 207 seals to the filter 3 by either a gasket or an o-ring 209 as one of ordinary skill in the art will understand. In one embodiment, a rubberized o-ring 209 is preferred, but other options include the following (or any combination thereof): a gravity seal (where the weight of the filter 3 seals it to the manifold 207), any type of sealant (e.g., a silicone caulk), any type of adhesive (epoxy, tape, and/or spray-foam), and/or a liquid or semi-liquid seal may be used.

The o-ring 209 may be used to maintain the pressure of the exhaust gas G₁ within the manifold enclosure 206 and provide a greater resistance to the gaseous flow. That is, since the manifold 207 surrounds the components 205 and seals them within the manifold enclosure 206, and since the housing 210 of the filter 3 is generally without substantial gas leakage, the gas leaked by the components 205 is captured in the enclosure 206, and mixed with the exhaust gas exiting from port 204 prior venting the mixed gas through the port 208. Therefore a majority of the 25% of gas that was previously lost without the manifold 207 is now reclaimed for use.

Note that the manifold 207 may be secured to the housing 210 by various fasteners, straps, screws and the like. However, in at least some embodiments, the manifold 207 is attached to the housing 210 in a manner whereby it can be attached and reattached without damage to the housing or the manifold so that the components 205 can be easily accessed when the manifold is detached from the housing 210.

In FIG. 12, the manifold 207 includes tabs and/or a rim 211 that assists in aligning and sealing the manifold onto the filter 3.

In one embodiment of the exhaust manifold 207, the manifold is used in conjunction with an oxygen separating air filter 3, wherein ambient air (or variation thereof) is taken in through the intake 201 and forced through the filter 3 with positive pressure, and oxygen is separated and exhausted through one exhaust port 203 and hypoxic air is exhausted through port 204. An embodiment of the manifold 207 can be provided for various commercially available air separation filters 3, such as: SeQual ATF Oxygen Concentrator Module part numbers—3161, 3428 3239, 2460, 2630, 1498.

The port 8 can be any type of fitting. Preferred embodiments are a standard glass filled nylon fitting, a nylon fitting, or a brass fitting. The exhaust fitting-mounting surface of port 208 protrudes from the manifold 207 wall to allow multiple orientations of the fitting.

Other features of the manifold 207 may include the following. A recess in the manifold material to allow wire routing past the o-ring 209 while still allowing the o-ring to maintain a seal to the filter 3. Multiple bolting patterns are integrated into the manifold 207 to allow the manifold and a variety of filters 3 to fit together.

In one embodiment, the manifold 207 may be integrated into a housing for an air separation unit 3. With this embodiment less injection molds may be required thereby reducing the complexity and cost of the entire system because there would be less parts for the combination of the filter 3 and the manifold 207.

4. Hypoxic and Normoxic Air Control Device for Altitude Simulation Systems

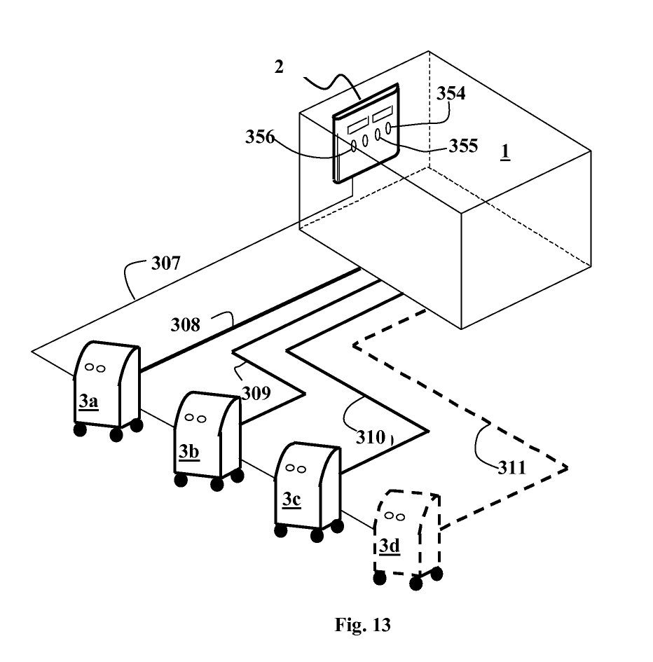

FIG. 13 depicts a hypoxic chamber 1, which includes a fixed structure, like a room or entire domicile, and/or a freestanding structure made of ridged or pliable material such as a tent. A hypoxic air generator(s) (e.g., generators 3a through 3d) supplies the enclosure 1 with a continuous source of hypoxic or normoxic air while the altitude simulation system disclosed herein is in use. Some altitude simulation systems according to present disclosure are open loop with a continuous air supply of normoxic or hypoxic air being forced through the system, while others are known as a closed loop system where the air is removed from the enclosure 1 via recirculation air feed lines (308, 309, 310 and 311) for supplying the system with re-circulated air from the hypoxic enclosure 1. Embodiments of the altitude simulation system depicted in FIG. 13 may be a blow-through system which is also known as an open loop or single pass system. Regardless of the type of system (e.g., open loop, or closed loop), multiple hypoxic air generators (e.g., 3a through 3d) can be used in any combination to supply the enclosure 1 with hypoxic air. The entire altitude simulation system may be controlled from the interior of the enclosure 1 via a controller 2 and data link 307 to communicate control signals to the hypoxic air generator(s) 3a through 3d.

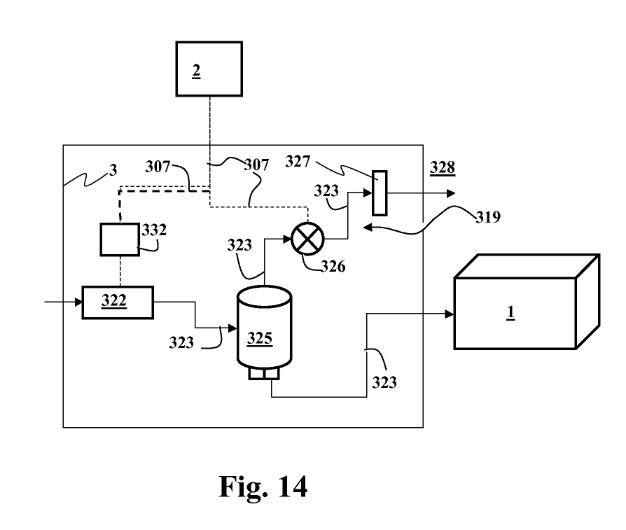

The control device 2 electronically controls the flow of air (hypoxic or normoxic) to the enclosure 1 in which a user(s) (human or mammal) resides for exposure to the simulated atmospheric environment therein. This control device 2) may be separate from the hypoxic air generators 3a through 3d (as shown in FIG. 13), or may be provided with one of the generators 3. Additionally, the controller 1 may be provided inside or outside the enclosure 1. In some embodiments, the controller 2 receives signals from the following sensors an oxygen sensor 354, a CO₂ sensor 355, and a pressure sensor 356) in the enclosure 1 for monitoring the atmosphere therein. The control device 2 electronically controls:
(a) the hypoxic air generators 3a through 3d,
(b) an oxygen valve system 319 (shown in FIG. 14 as outside of the enclosure 1 and integrated into a generator 3. Note, an embodiment of oxygen valve system 319 may be located in each of the hypoxic air generators 3a through 3d.
(c) other devices that may include venting fans, re-circulators and/or any device that may be used to allow the input air into the air generators 3a-3d to be fully or partially supplied from the air from the enclosure 1, and
(d) any CO₂ scrubbers (not shown) for reducing the CO₂ in the enclosure 1.

The controller 2 can have three or more separate channels to the generators 3a-3d that it controls; only one channel 307 is depicted in FIG. 13. Note the a channel includes a single or a series of hypoxic air generators 3 and/or additional devices all controlled by the same control signal from the controller 2. More generally, hypoxic air generating units (e.g., generators 3, air scrubbers, nitrogen generators, etc.) may be used individually or in multiples, and the units can be linked in series to an embodiment of the control device 2 thereby eliminating the need for a control channel 307 for different air generating units 3 that are controlled by the controller 2. However, multiple control cables 307 may be required for multiple channels. Although in one embodiment, the controller 2 and the air generating units 3 may communicate wirelessly, e.g., via Bluetooth or another wireless protocol.

The oxygen valve system 319 depicted in FIG. 14 switches between providing hypoxic air and providing normoxic air as a response to a signal from the controller 2 on the channel 307. This oxygen valve control signal also may be transmitted by the controller 307 on additional (if any) channels for controlling other oxygen valve systems 319. Each hypoxic air generator 3 houses a compressor/motor 322 that receives air from the ambient environment and compresses it to pressurize a corresponding embodiment of the oxygen valve system 319. The compressed air flows through tubing, ducts or vents 323 to the molecular sieve beds 325. Within these molecular sieve beds 325 oxygen is partially extracted from the air provided by the ducts or vents 323 resulting in two products: (a) hypoxic air, and (b) oxygen or oxygen rich air (i.e., air with greater than 20.9% O₂). The oxygen valve system 319 includes a servo-controlled valve 326 that may be closed in the default (i.e., non-actuated) state, and open or partially open in its actuated state. When the servo valve 326 is actuated by a signal from the controller 2 via the channel 307, the oxygen line from the molecular sieve beds 325 is free to flow through a filter 327 into the ambient environment 328 while hypoxic air flows into the hypoxic enclosure 1. If the servo-controlled valve 326 is closed, then the oxygen cannot flow from the molecular sieve beds 325 rendering them inoperable at that time and thus allowing only normoxic air to flow from the molecular sieve beds 325, and accordingly flow through the oxygen valve system 319 to the enclosure 1. As an added benefit of the oxygen valve system 319, it forces its hypoxic air generator 3 to only flow normoxic air in case of controller 2 and/or valve 326 failure. That is, there is a biasing safety mechanism (not shown in the figures) that closes the valve 326 when, e.g., no signal is received from the controller 2 or a signal from the controller is detected, but the valve 326 does not respond appropriately. Such a biasing safety mechanism may be a valve that is controlled via a solenoid where the solenoid valve is closed in the powered-off position and therefore forcing only normoxic air through the system. This failsafe safety feature prevents an unsafe atmosphere within the enclosure 1 (e.g., an atmosphere having an unsafe concentration of CO₂). This failsafe feature may be additionally triggered by excessively high temperature, excessively high CO₂ levels, and/or excessive hypoxia within the enclosure 1.

The controller 2, via the channel 307, turns on and off the air generating unit(s) 3 (e.g., hypoxic air generators 3a-3d) by activating a relay 332 (FIG. 14) in each unit, wherein the relay completes a power circuit (not shown) for the compressor/motor 322. The controller 2 has associated therewith circuitry providing a programmed system delay to prevent tripping a circuit breaker (not shown) that is built into each hypoxic air generating unit's power system for preventing the hypoxic air generator from drawing too much amperage for the external power source (e.g., a residence, training facility, or hospital room). After shutdown of an air generating unit 3, its oxygen valve system 319 may be still pressurized. The startup amperage required by the electric motor of the compressor 322 may be high enough from a backpressure in the oxygen valve system 319 to cause the circuit breaker to be tripped. Accordingly, the controller 2 waits a predetermined amount of time before restarting each air generating unit 3. Such a predetermined amount of time may be dependent on the length of time required for such backpressure to dissipate. In at least one embodiment, this predetermined amount of time is in the range of 10 to 60 seconds, and more typically in a range of 20 to 40 such as 30 seconds. Additionally note that the controller 2 does not turn on all of the air generating unit(s) 3 connected to it concurrently, but instead staggers their activation. This staggering reduces the start-up current needed from an electrical supply for activating an embodiment of the altitude simulation system disclosed herein. Appendix F provides additional details regarding a particular embodiment of the circuitry providing a programmed system delay.

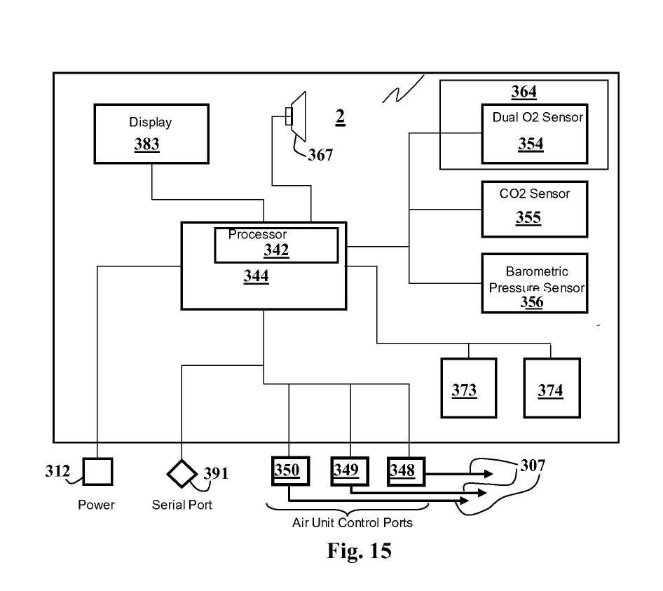

FIG. 15 is a schematic of the components of the controller 2. A microprocessor 342 is operably connected to a main circuit board 344 of the controller 2. The processor 342 uses an algorithm based on the West Equation (see Appendix E) to control the hypoxic generators 3 via the one or more control link ports 348-350 which are each, in turn, operably connected to a channel 307. It is important to note that the arrangement depicted in FIG. 15 shows a three-channel controller 2 having one main control link port 348 and two supplemental control link ports 349, 350 for controlling air generating units 3 via three different channels 307. However, any given controller 2 may have more or less control link ports. The processor 342 gathers data from a minimum of three different sensors (e.g., one or more oxygen sensors 354, a CO₂ sensor 355, and a barometric pressure sensor 356). The controller 2 has slots available for one or more additional sensors such as a sensor for an ammonia and/or other volatile compound (e.g., any type of compound that may be deemed harmful to the user in concentrated quantities). Accordingly, the output for such additional sensors can be used by processor 342 to further ensure the safety and reliability of the altitude simulation system disclosed herein.

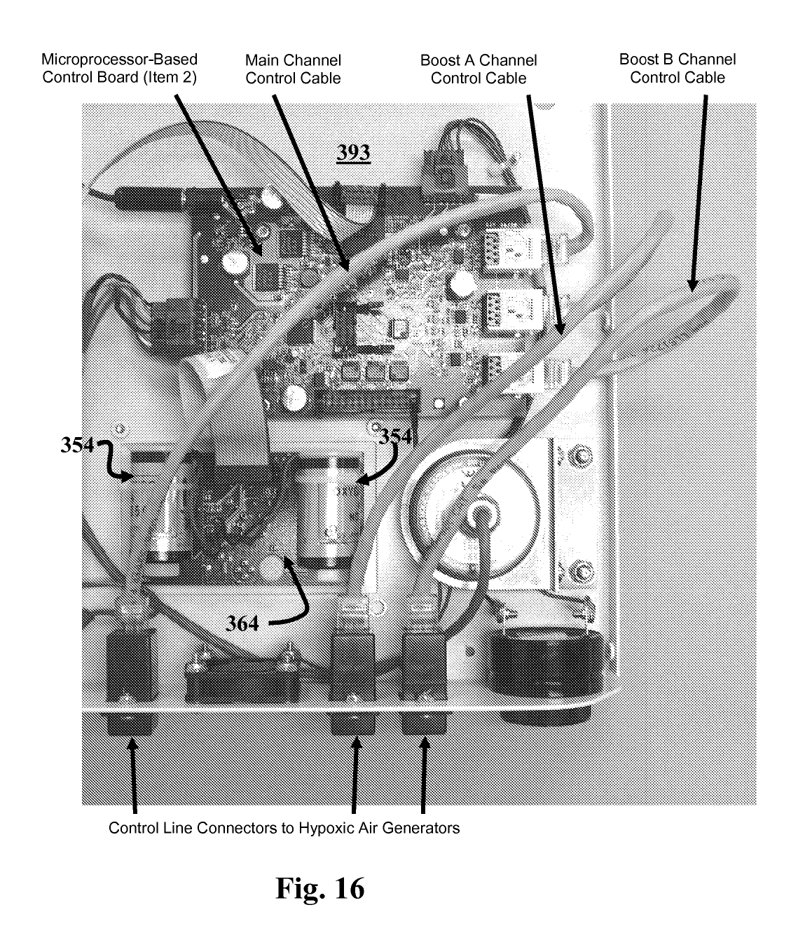

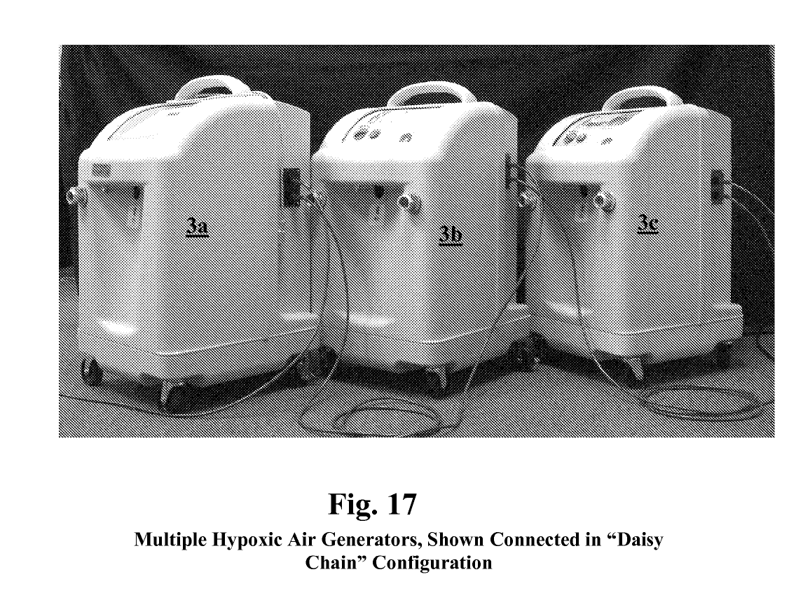

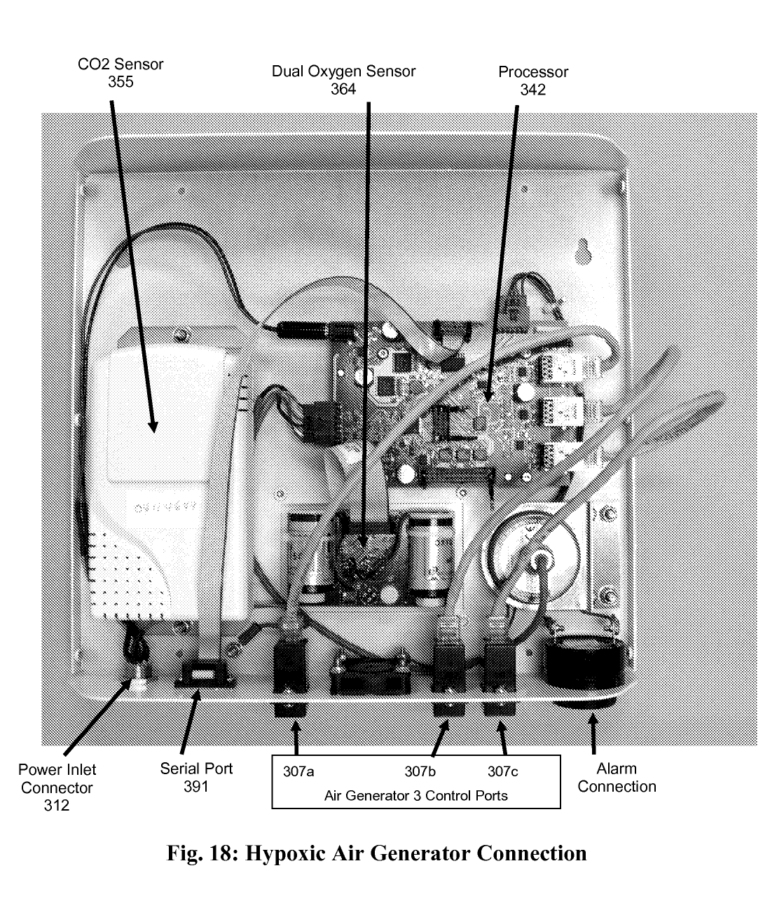

The sensor(s) 354 may be a pair of two oxygen sensors on a dual oxygen sensor circuit board 364 (FIGS. 15 and 16), which may be used to measure the level of oxygen in the air that is contained in the enclosure 1. The second sensor 355 includes a carbon dioxide sensor that is used to measure the level of carbon dioxide in the air within the enclosure 1. The processor 342 uses the data from this sensor 355 to determine if unacceptable levels of carbon dioxide are reached inside the enclosure 1. If a predetermined upper limit of carbon dioxide concentration is reached, then the controller 2 sounds an audible alarm 367 to notify a user that the carbon dioxide levels inside the enclosure 1 have become unsafe. The third sensor used by the processor 342 includes a barometric pressure sensor 356 that determines the actual altitude of the ambient environment (e.g., the altitude of the enclosure 1 before a simulated altitude is introduced into the enclosure). Additional sensors that can be added include (but are not limited to) a temperature sensor 373 and a humidity sensor 374.

Output from the processor 342 may be visible on the display panel 383. For example, the following information can be displayed on the display 383: an actual altitude of the enclosure 1, the current simulated altitude within the enclosure 1, CO₂ levels within the enclosure 1, etc. The functions and readings on the display 383 can be seen in the operating manual that is provided in Appendix A hereinbelow. A serial port 391 is connected to the processor 342 for programming and diagnostic purposes.

Other features of the controller 2 include the following:
(a) The digital readout 383 displays the simulated altitude based the oxygen level in the enclosure 1. Since the concentration of the oxygen in the enclosure 1 can fluctuate due to several factors, including but not limited to sensitivity of the sensors (e.g., the oxygen sensor, the carbon dioxide sensor, and/or the barometric pressure sensor). Of all these sensors the output voltage of the oxygen sensors shows the greatest fluctuation. (e.g., the sensors 354), ambient temperature, ambient humidity, and changes in atmospheric pressure, an averaging technique may be employed to average simulated altitude values thereby to avoid nuisance changes in the calculated simulated altitude. In particular, the averaging may be performed on a moving window of consecutively calculated simulated altitude values, wherein the window may be, e.g., for 50 to 100 such values. In addition, when the enclosure 1 reaches the desired predetermined simulated altitude (e.g., a “set point” as one of ordinary skill in the art will understand), the display 383 displays this set point value as the simulated altitude as long as the calculated simulated altitude is within ±200 ft of the set point.
(b) The processor 342 can be programmed for automatic power on/off based on the programmed settings that are inputted by a user. For example, such a feature is useful when the user wants the present altitude simulation system to be at simulated altitude as soon as the user gets home (e.g., from work, etc.) since it may take time for the system to reach the desired simulated altitude, and not all users run the altitude simulation system during the day. In addition, the processor 342 executes programs that can learn from historical on/off time data to find cyclical patterns and use that data to power itself on/off at the times that it has typically been turned oil or off in the past. Of course, such a feature is activated only by a user explicitly requesting such activation. However, such a feature provides user convenience and helps assure that a desired simulated altitude treatment or schedule is provided on a consistent and reliable basis. Such learning, in one embodiment, is performed by the processor 342 performing a program that averages each of the start time, the shut off time, and the desired simulated altitude over a given period of time (e.g., 30 days), and then using the resulting average values as defaults, unless such values are manually overridden by the user.
(c) Due to fluctuations within the enclosure 1 of the barometric pressure, sensor sensitivity, ambient temperature, ambient humidity, random error, and systematic error, the controller 2 may not display the correct simulated altitude for the known location of the ambient actual altitude of the enclosure 1. To correct this problem, the controller 1 can be programmed to use a minimum altitude reading from which calculations of simulated altitudes are derived. This minimum altitude may be set for the known average altitude of a region containing the installation location, but can be set for any altitude that is safe for a user in the enclosure 1. For example, if it is known that the altitude simulation system is to be installed in the metropolitan area around Denver, Colo., U.S.A., then the default attitude may be set for 5,000 feet. Note that in at least some embodiments, the minimum altitude can not be set to be approximately more than 2,000 to 4,000 feet in altitude above the altitude that has been determined for enclosure 1 surrounding site.
(d) The controller 2 may use barometric pressure measurements to further ensure that a correct and/or allowed ambient altitude value is being used as the minimum altitude.
(e) The controller 2 may be self-calibrating to insure continued accuracy over the life of the oxygen sensors 354 since such sensors tend to degrade in measurement accuracy and/or reliability with age. After a specified time that is dependent on the type of oxygen sensor 354 used, the altitude simulation system will recalibrate such sensors. For example, after an interval of 35 days, the controller 2 may automatically recalibrate the sensor(s) 354 when the altitude simulation system is put in standby mode (i.e., turned off for the day). If the altitude simulation system reaches, e.g., 42 days without recalibration of the sensor(s) 354, then the controller 2 forces the altitude simulation system to recalibrate such sensors regardless of any usage of the altitude simulation system during the 42 days. When the controller 2 is (re)calibrating, it turns on all hypoxic air generators 3 on all channel(s) 307, but keeps the oxygen valves 319 closed in order to fill the enclosure 1 with normoxic air. In addition, any available exhaust devices are actuated at that time for circulating surrounding ambient air into the enclosure 1. The purpose for this procedure is to bring the oxygen level in the enclosure 1 to that of ambient air which contains 20.94% oxygen. The controller 1 then calculates new calibration coefficients that are then used to compute subsequent simulated altitude values that have a greater assurance of being appropriately accurate (e.g., having an oxygen partial pressure corresponding to an altitude of within 200 feet of a desired simulated altitude).
(f) The controller 2 may perform an upper limit calibration (also referred to as a high altitude refinement) for ensuring more accurate readings of the simulated altitude in the enclosure 1. The technique requires that a high altitude be simulated by running the altitude simulation system up to a maximum expected simulated altitude. Then, the oxygen level in the enclosure 1 is measured, using an independent sensor (e.g., an additional sensor 354). The measured value may be programmed into the controller 2 for higher accuracy in operating ranges that simulate high altitudes (e.g., between 9,000 feet and 12,000 feet). For example, 15% to 20.94% oxygen.

As described above, the controller 2 may employ two oxygen sensors 354 that are used to measure the oxygen levels within the enclosure 1. One of these sensors 354 supplies the processor 344 with the required information on the oxygen concentrations in the enclosure 1 to thereby allow adjustment of controller 2 parameters used to keep the actual system value in equilibrium within a particular range of the set point value (e.g., within ±200 feet of altitude). The output from the second sensor 354 bypasses the processor 342 and may be transmitted directly to the hypoxic air generator oxygen valve safety mechanism discussed above via an analog circuit on the circuit board 344 of the controller 2. Thus, the second sensor 345, the analog circuit, and the safety mechanism at a hypoxic air generator's oxygen valve 319 provides a redundant safety feature that forces the altitude simulation system to deliver normoxic air to the enclosure 1 if the oxygen in the enclosure is outside of specified safety limits (e.g., such limits being less than 15% oxygen in the enclosure 1).

Additionally note that the output from both of the oxygen sensors 354 also are compared with one another to further ensure that both sensors 354 are working within the specified limits of one another. For example, if the output of the sensor 354 used by the processor 342 is not in equilibrium with the duplicate sensor 345 (e.g., the output voltages from the two sensors are not within at least 5% of one another), then the altitude simulation system (more particularly, the controller 2) displays an error message and shutdowns. In one embodiment, immediately prior to such a shutdown, the controller 2 may force the generator(s) 3 to input normoxic air into the enclosure 1 for a predetermined amount of time (e.g., 5 minutes) as a safety measure.

The oxygen sensors 354 currently commercially available typically need to be replaced periodically. In one preferred embodiment of the simulated altitude system, the oxygen sensors 354 should be replaced approximately every year. In one embodiment, an access panel 393 (partially shown in FIG. 16 and more fully shown in Appendix C) for accessing the controller 2 also has the sensors 354, 355, and 356 mounted thereon as well. Note that the access panel 393 allows a user to easily replace the dual oxygen sensing board 364 having the oxygen sensors 354 thereon. Moreover note that the dual oxygen sensing board 364 stores the lifetime usage of the sensors 354 and transfers this information through the processor 342 to the display 383. The processor 342 alerts a user via the display 383 when the oxygen sensors 354 need to be replaced, and renders the altitude simulation system inoperable if the sensors 354 are not replaced in a predetermined time frame of, e.g., one year of use.

An “indefinite system shutdown” feature may be built into the controller 2. This feature prevents a user from using the altitude simulation system if there has been a breech of contract with the vendor (i.e., failure to pay).

In one embodiment, the altitude simulation system uses 24 VDC as a power supply throughout. This permits usage of sensors, fans, relays and other input/output devices that use this voltage without modification. It also reduces current flow in connected control cables from what would be needed at other voltages, e.g., greater than 24 Volts

In another embodiment of the altitude simulation system, the controller 2 measures volatile organic gaseous compounds such as methane and ammonia. When unacceptable or dangerous levels of these compounds are detected inside the enclosure 1 by the controller 2, mitigating actions can be taken to remove them. For example, the controller 2 may initiate the following activities: turning on an exhaust fan(s) for venting the enclosure 1, and turning off oxygen valve system 319 to thereby introduce normoxic air into the enclosure 1 and thereby remove dangerous gaseous compounds.

In one preferred embodiment of the altitude simulation system, a Global Positioning System (GPS) receiver is utilized to determine an installation site altitude for an installation of the altitude simulation system. In this embodiment, when the altitude simulation system is relocated, the controller 2 will acquire information from a GPS module to determine the new altitude, and use the new altitude to determine how to simulate altitudes within the enclosure 1.

In another embodiment of the altitude simulation system, the controller 2 may be programmed with the altitude to which it is being shipped. In this embodiment, it is necessary to reprogram the installation altitude if the installation site changes.

Oxygen Sensors 354

Currently there are three types of oxygen sensors 354 available for use with the controller 2: electrochemical sensors, catalytic bead sensors, and paramagnetic sensors. Electro-chemical sensors are currently used in one preferred embodiment of the altitude simulation system. The chemicals in these sensors get consumed as the sensor is used, and therefore give them a limited life of approximately one year. Also, the consumption of the chemicals causes the sensors to vary their output over time. Thus, there is a need for the controller 2 to perform a recalibration as described hereinabove. Alternatively, the sensors 354 may be either the catalytic bead or paramagnetic sensors. These sensors are much more stable and are less temperature sensitive than the electro-chemical sensors. In addition, they have little sensitivity to pressure changes. Catalytic bead sensors have a finite life while the paramagnetic sensors have a substantially infinite lifespan as long as they are not subjected to extreme mechanical vibration or particulates (since they include mechanical components). Any type of oxygen sensor can be integrated into the controller 2 as long as the sensor's circuit output can be conditioned to be between 0 and 2.4 VDC. Alternative embodiments of the controller 2 can use the catalytic bead sensor FCX-U which is manufactured by Fujikura America Inc. having an address of 3001 Oakmead Village Drive, Santa Clara, Calif. 95051-0811 (408-748-6991). These sensors need to be heated slowly (2-3 minutes) in order to not damage the bead. The catalytic bead needs to be heated to approximately 300° C. The power supply in the controller 2 must supply approximately 3 watts for each of the 2 catalytic sensors 354 for this heating. Additionally, the software for the processor 342 requires modification if the catalytic bead sensors are used because their signal output is logarithmic. Moreover, the output from such catalytic bead sensors is indeterminate until they reach operating temperature. Therefore to avoid fluctuation in, e.g., the display 383 during the first 2 minutes after power-up, the software for the processor 342 needs to be programmed for a 2-3 minute delay after power-up.

5. Hypoxia as a Treatment for Certain Medical Conditions

Additional sensors may be provided in an embodiment of the altitude simulation system for measuring the oxygen content in a user's blood and provide inputs from such a sensor to a controller (e.g., controller 2) that controls hypoxic air generators 3 delivering hypoxic air to the user via the enclosure or a mask. In the present embodiment, the additional sensors include an oximeter connected to the user. The oximeter may operably communicate with the controller 2 via a wire or wireless link for controlling the gas mixture supplied to the user. Redundant or fail safe hardware and/or algorithms provide user safety, wherein the user may be allowed to input set points to the controller 2 within safe limits (e.g., within a blood oxygen saturation range of 85% to 100%).

6. Means of Providing Hypoxic Air and Normoxic in Hypoxic Altitude Simulation Systems

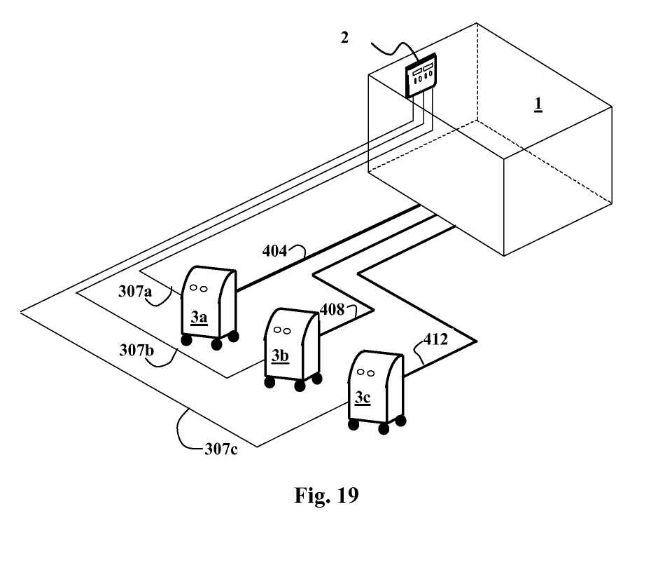

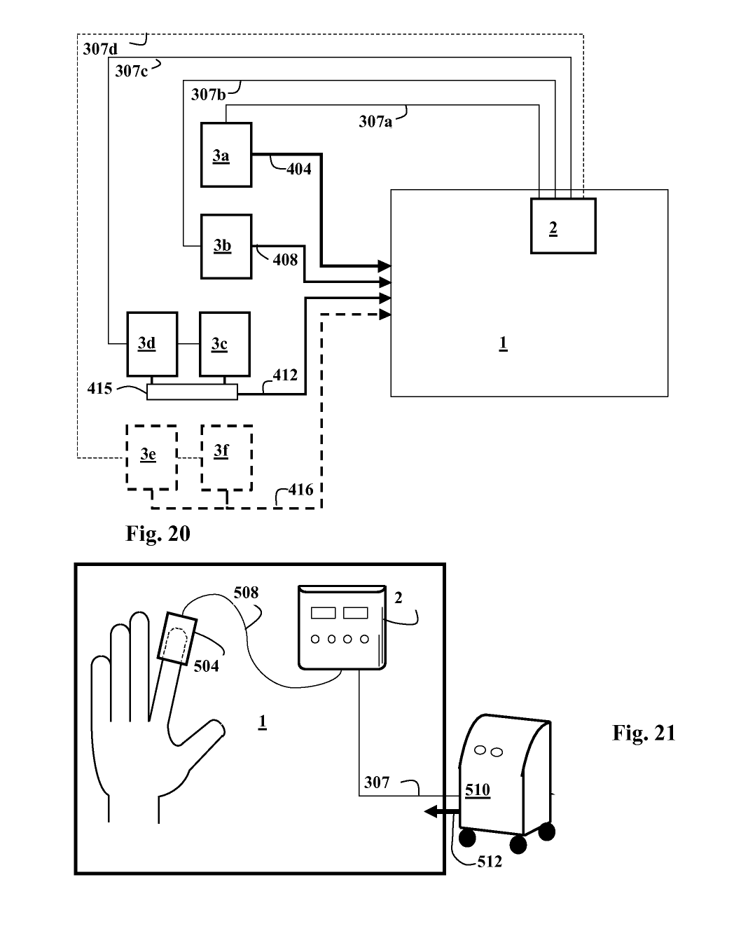

Referring to FIGS. 19 and 20, a controller 2 includes a means for setting a set point (i.e., a desired simulated altitude or oxygen saturation for the enclosure 1). The set point may be in terms of an O₂ percentage, a simulated altitude, or an arterial oxygen saturation. The controller 2 controls multiple hypoxic air generators (e.g., 3a, 3b, and 3c in FIG. 19, although more or less generators may be utilized). Note that each of the hypoxic air generators 3a, 3b, and 3c has a control link with the controller 2. The controller 2 operates a main control communication channel via the main channel control link 307a connected to the first hypoxic air generator 3a for controlling this generator. Each of the other hypoxic generators 3b and 3c also has a respective control link 307b and 307c for receiving control signals from the controller 2. Each of the hypoxic air generators 3a, 3b, and 3c provides air (hypoxic or otherwise) to the enclosure 1 by way of a corresponding one of the feed lines (404, 408, 412). The controller 2 operates boost channel A (including control link 307a, generator 3a, and feed line 404) to provide hypoxic (or otherwise) air to the enclosure 1 via feed line 404. Similarly the controller 2 operates a boost channel B (including control link 307b, generator 3b, and feed line 408) to provide hypoxic (or otherwise) air to the enclosure 1 via feed line 408. Additionally, the controller 2 operates a boost channel C (including control link 307c, generator 3c, and feed line 412) to provide hypoxic (or otherwise) air to the enclosure 1 via feed line 412. Note that each such channel may not be limited to a single hypoxic air generator 3 as shown in FIG. 19. However, FIG. 20 shows an embodiment of a boost channel having two hypoxic air generators 3c and 3d controlled by signals from the one control link 307c. That is, this boost channel includes control link 307c, generators 3c, 3d, and feed line 412. Note that the air lines from the generators 3c and 3d are connected via an intersection or manifold 415 and then a single feed line 412 from this manifold outputs air to the enclosure 1. In addition, an altitude simulation system may not be limited to only three such channels; the system may have more or less such channels. For example, FIG. 20 shows an additional boost channel including control link 307d, generators 3e, 3f, and feed line 416) to provide hypoxic (or otherwise) air to the enclosure 1 via feed line 416.

Once the enclosure 1 is at a desired simulated altitude known to the controller 2, all boost channels may not be required to be actively providing air to the enclosure 1 for maintaining the simulated altitude therein. Therefore one or more of the channels can have their generators 3 deactivated for quieter, cooler, and more economical operation of the altitude simulation system. The first hypoxic air generator 3a may be the primary unit that is constantly generating hypoxic air while the altitude simulation system is in use in order for the controller 2 to maintain the desired simulated altitude in the enclosure 1. When the oxygen concentration in the enclosure 1 deviates sufficiently from a certain level (e.g., 20% above or below a desired set point), or the CO₂ concentration within the enclosure 1 rises above desired levels, one or more of the boost channels can be activated by the controller 2 for rectifying the deviation. That is, activating such additional boost channels cause the altitude simulation system to produce more hypoxic air. One or more, or all of the boost channels can be activated by the controller 2. For example, the controller 2 may activate the boost channels B and C to return the enclosure 1 to the desired simulated altitude when actual conditions vary significantly from a current set point. For example, if the oxygen concentration in the enclosure rises above 15% of the controller's set point, then boost channel B may be activated, and if CO₂ concentration within the enclosure 1 rises above desired levels, then both of channels B and C may be activated.

7. Oxygen Control via Pulse Oximetry

Prior art altitude simulation systems regulate oxygen concentrations in enclosures 1, masks 101. Generally these altitude simulation systems regulate oxygen by providing hypoxic air, normoxic air, or hyperoxic air depending on the desired simulated altitude and/or desired level of oxygen. However, a user's arterial oxygen saturation can be used to determine an appropriate hypoxic air and/or normoxic amount of air to be supplied to the user.

Controlled oxygen concentrations in an enclosure may be monitored in order to determine their affect upon arterial oxygen saturation since arterial oxygen saturation is the key element to triggering the body's response to hypoxia. However, different users respond differently to the same concentration of oxygen in the air. For example, person A may have an arterial oxygen saturation of 89% while breathing air that is 14% oxygen while person B, breathing the same air, might have an arterial oxygen saturation of 92%. Since different individuals may have different arterial oxygen saturations in response to the same oxygen concentration in air, there is a need to regulate the amount of oxygen which an altitude simulation system supplies to a user based at least in part on measurements of arterial oxygen saturations.

Non-invasive pulse oximetry is well known for measuring arterial oxygen saturations. Pulse oximeters are used to determine the level of oxygen saturation in arterial blood. Readings are generally in terms of a percentage of oxygen saturation in the blood. However, heretofore pulse oximeters have not been used to directly regulate the amount of oxygen in controlled environments (e.g., an hypoxic enclosure 1). Since arterial oxygen saturation may be the key to triggering the body's mechanisms for adapting to altitude (i.e., a lower or higher concentration of oxygen in air), the use of a pulse oximeter provides a more exact means of determining when a user's adaptive mechanisms to changes in air oxygen content will be triggered.

FIG. 21 illustrates the use of a pulse oximeter 504 (or other similar device) for measuring the arterial oxygen saturation in a user's hand. The readings from the pulse oximeter 504 are used as an input to a controller 2 via a cable or wireless connection 508. The controller 2 uses such readings for controlling an oxygen concentrator 510 (or, other oxygen sources, or sources of hyperoxic air (where O₂>20.94%), or nitrogen generators, other sources of N₂, or sources of hypoxic air). The desired target arterial oxygen saturation may be determined by a user (e.g., within an enclosure 1 supplied with an air flow 512 having a modified oxygen content), and entered into the controller 2. The controller 2 uses the reading from the pulse oximeter 504 or other such device to regulate the oxygen concentrations within the enclosure 1 by controlling an oxygen concentrator(s) 510 or nitrogen generators or other such devices. When arterial oxygen saturations are too low relative to desired levels, the controller 2 turns off or reduces the hypoxic air flow 512 to the user and/or increases a normoxic or hyperoxic air flow 512. When arterial oxygen saturations are higher than desired, a hypoxic air flow 512 is increased in either volume or in terms of greater hypoxia.

Hypoxic air flow may be delivered to an enclosure 1 that encloses the user or to a breathing mask that delivers air or hypoxic air or hyperoxic air to the user. Thus, more or less oxygen can be made available to the user depending on the desired level of oxygen saturation detected in the user's blood.

The term “airflows” in this document refer to air that may be normoxic—approximately 21% oxygen, hyperoxic—higher than 21%, or hypoxic—lower than 21%. The term “air unit” or “air generator” herein refers to an air separation or concentration unit that employs a technology such as zeolite and/or pressure swing adsorption or other technology to separate air into a hyperoxic air flow and and/or a hypoxic air flow.

A pulse oximeter 504 may be attached to a user (e.g., to a finger of the user). The pulse oximeter 504 measures blood oxygen saturation in the user. The pulse oximeter sends a signal based on the user's blood oxygen saturation to a controller 2. This electrical signal can be used as an input to the controller 2 for controlling air units 510 so that such air units provide different oxygen concentrations in the air flow 512 to an enclosure (or mask) from which the user breathes. In particular, the controller 2 uses such readings of the user's actual arterial oxygen saturation and a target or desired oxygen saturation for determining how to vary the concentration of oxygen in the airflow 512. The controller 2 allows the user to set a desired oxygen saturation target (e.g. 89%). The controller 2 is in signal communication with the air unit(s) 510 that provide air flow 512 to the user for breathing. The software and hardware of the controller 2 turn on and off the air unit(s) 510 which may be configured to provide normoxic air (approximately 21% oxygen), hypoxic air (<21% oxygen) or hyperoxic air (>21% oxygen). By modulating airflows from the air unit(s) 510 or by turning an air unit(s) 510 off and on, the controller 2 can provide different ambient concentrations of oxygen to the user which in turn affects the user's arterial oxygen saturation.

Since oxygen saturations vary widely over the course of time—especially during sleep, signal averaging may be useful to determine trigger points that turn on airflows 512. In addition very high short duration readings or low short duration readings may be discounted by the controller 2 software when determining the control of airflows 512.

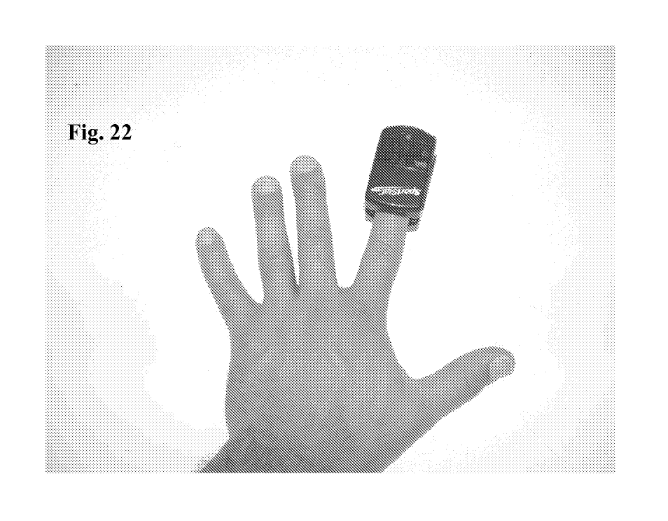

FIG. 22 shows a wireless embodiment of a pulse oximeter 504. The pulse oximeter is manufactured by Nonin Medical Inc. located at 13700 1st Avenue North, Plymouth, Minn. 55441-5443, U.S.A. (763-553-9968) Note that this pulse oximeter shows the user his/her pulse rate as well as the oxygen saturation in the user's blood.

8. Enclosure 1 Fan and Damper Control

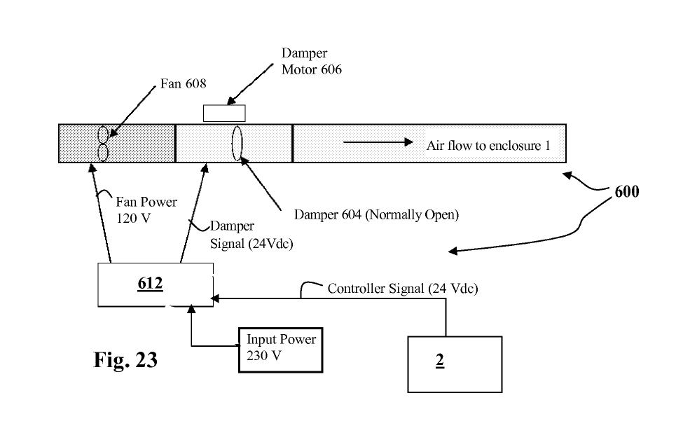

FIG. 23 shows an illustration of a ventilation subsystem 600 including a damper 604, a damper motor 606 for opening and closing the damper, fan 608, and a fan/damper controller 612, wherein the present configuration is particularly suited for large enclosures 1 having potentially 20 to 50 people therein. The fan/damper controller 612 receives a signal from the controller 2 and activates the fan 608 as needed. The damper 604 closes when the fan 608 is not operating so that the leakage from the enclosure(s) 1 is reduced.

The ventilation subsystem 600 operates on a 24 Volt dc signal from the controller 2. Based upon such a signal, relays in the fan/damper controller 612 either apply power to, or remove power from the fan 608 and the fan damper motor 606. The fan 608 operates on 120 Vac and the damper motor 604 operates on 24 Vdc. The damper 604 is normally open, so that it will remain open if the signal from the controller 2 is lost or if power is interrupted. Similarly, the relays for the fan 608 will be normally closed so that power is supplied to the fan if the signal from the controller 2 is lost. This provides failsafe operation for both the fan 608 and the damper 604. The following table lists the states for the ventilation subsystem 600.

| Input Signal from | Output to the | Output to the |
|---|---|---|
| the Controller | Fan motor | Damper |
| 0 | 120 Vac | No Power |
| 1 | No Power | 24 Vdc |

The foregoing description has been presented for purposes of illustration and description. However, the description is not intended to limit the invention as claimed hereinbelow to the form disclosed hereinabove. Consequently, variations and modifications commensurate with the teachings, within the skill and knowledge of the relevant art, are within the scope of the claims hereinbelow. The embodiments described hereinabove are further intended to explain the best mode presently known of practicing the invention claimed hereinbelow, and to enable others skilled in the art to utilize the claimed invention in various embodiments, and with the various modifications required by their particular application or uses of the invention. Thus, it is intended that the appended claims be construed to include alternative embodiments to the extent permitted by the prior art.

### APPENDIX A: USER'S GUIDE AND SYSTEM INFORMATION

A-1 System Overview

This guide contains lots of useful information for installation and operation of your system. It also contains important safety information. The safety information is formatted to stand out, and is preceded by the word “WARNING”. This safety information is very important to your safe use of this product. Please read and pay extra attention to the warning statements.

Your Colorado Altitude Training system or Colorado Mountain Room consists of the following components, which arrived in several boxes:

Air Processing Units: You received between 1 and 6 air units for your installation depending on the size of your room and options that you ordered with it. These are all individually boxed.

Tent or Clear Room: You received either a tent (packaged in two boxes) or a clear room (packaged in many boxes).

Scrubber: Your system may contain a Carbon Dioxide (CO2) scrubber. If it does, you received “SOFNOLIME™ CO2 Absorbent” to “charge” your scrubber with.

Control Module: Your control module is the heart of the altitude simulation system. It allows you read the simulated altitude of your room, and set it to the desired value. It connects to and controls air units, scrubbers and vents.

Accessories: Included with your system is one or more of the following accessories:
Air Delivery Tubes
Control cables for air units, vents and scrubbers
Information Kit containing an Altitude Chart and Altitude Disk
Packaging Checklist from the CAT Quality Assurance Department
   A-2 Setting Up Your Colorado Mountain Room Controller

Please refer to the Colorado Mountain Room Installation Guide, Part Number 1290, for information on setting up your system.

A-3 Daily System Operation

A-3.1 First Time Operation:

Note: If Colorado Altitude Training installed your system for you, you may skip to “System Overview” below.

Before applying power to your controller for the first time, make sure that you have connected all of your air units, and scrubber (if equipped) to the controller as described in your Colorado Mountain Room Control System Installation Guide.

Please plug the power supply into power at this time. The system will activate, turn itself on and take a variety of measurements. It may go into a calibration mode, denoted by the words “OPEN door” on the display, followed by the word “CAL” on the display. If this occurs, it is normal. When the unit is done calibrating, it will return to the STANDBY mode or it will go into the ON mode, depending on the position of the ON/STANDBY switch.

If your system returned to the Standby mode, you may now turn your system on. The display will go through a self check process, and then the controller will turn on all of the air units connected to it. At this time, it is important to check each of the air units connected to the controller to confirm that they are adjusted properly. Read the flow valve on the front of EACH of the air units to make sure that the flow rate is 10 Liters Per Minute (LPM). Adjust the flow control knob next to the meter if required to achieve a flow of 10 LPM. When you are done adjusting the flow control valves, turn your controller off again. Your system is now ready for use.

A-3.2 System Overview

Your Colorado Mountain Controller is a sophisticated, microprocessor based system. The microprocessor reads the electrical signals coming from two oxygen sensors, a carbon dioxide sensor and an atmospheric pressure sensor. It uses this information to calculate the simulated altitude, and in turn to control air units, scrubbers and vents as needed to maintain your room at its simulated altitude setting.

A-3.2.1 Control and Displays:

[image]

1. Power Switch: this control toggles back and forth between the “ON” state and the “STANDBY” state.

2. Feet/Meters Switch: This control toggles back and forth between displaying altitude set points and simulated altitude reading in feet or meters.

3. Actual Altitude Display: This window displays the current simulated altitude of the system as measured and computed by the controller. It can be displayed in feet or meters. The reading in this display will vary with the atmospheric pressure at the location, which varies with weather. Hence, it is normal for this number to change somewhat from day to day. You may notice a drift in the Actual Altitude display. If this occurs, perform a manual calibration oil your system at your earliest convenience. See below for instructions on how to do this.

4. Set point Altitude Display: This window displays the current simulated altitude set point as selected by the user. The set point has a range from 1000 feet to 15,000 feet, in increments of 100 feet or 25 meters. The current set point can be displayed in feet or meters. The system remembers the setting from day to day, so there is no need to re-set it each time you turn your system on.

5. Altitude Set Point Adjust Switch: This control increments or decrements the altitude set point. Press and hold the top portion of the switch to increase the altitude set point. Release the switch when the desired simulated altitude set point is shown on the display. Press and hold the lower portion of the switch to decrease the altitude set point.

6. Set point/CO2 Switch: In normal operation, the set point window is displaying the current set point. However, your controller can display the Carbon Dioxide as measured in the room if desired. To show the CO₂, depress the top of the Set point/CO₂ switch. The CO₂ will be displayed in the set point window in Parts Per Million (PPM) as long as the switch is depressed. Releasing the switch reverts back to displaying the set point altitude again.

7. Brightness Control: This control varies the brightness of the control panel, including both the display windows and the indicators. Turning this control clockwise increases the brightness, counter-clockwise decreases the brightness.

8. Audible Alarm (not shown in diagram above): The controller will sound an audible alarm if the simulated altitude reaches an unacceptable level. If this occurs, the system should be turned off. DO NOT attempt to use your system again. Open the doors to the room to allow the room to return to normal altitude. Contact CAT customer service for assistance at 1-877-258-4883.

The controller also sounds the audible alarm when the level of CO₂ in the volume has reached an unacceptable level and is not being successfully mitigated by the scrubber or vent. This alarm will silence once the CO₂ level in the volume has returned to an acceptable level. Opening the doors to the volume is the quickest way to clear the CO₂ levels.

A-3.2.2 Basic Operation:

To turn on and operate your controller, perform the following:
1. Switch the power switch from the STANDBY state to the ON state.
2. Select Feet or Meters for your displays.
3. Select the desired simulated altitude for your room. Remember to start out at an altitude that is only a few thousand feet (about 1000 meters) above your actual altitude.
4. Allow the system to operate.
5. After one-two weeks at your initial simulated altitude, you can begin to gradually increase your simulated altitude set point. The recommended rate is approximately 1000 feet per week. Consult your CAT salesperson for guidance on increasing your simulated altitude over time.

A-3.2.3 Features:

Your Colorado Mountain Room contains many features. These features are explained below:

A-3.2.3.1 Auto-Calibration:

IT IS VERY IMPORTANT THAT THE ROOM NOT BE AT SIMULATED ALTITUDE DURING THE CALIBRATION PROCESS. OPENING ALL AVAILABLE DOORS AND WINDOWS WILL SPEED UP THE CALIBRATION PROCESS.

Your Colorado Mountain Room needs to recalibrate itself periodically in order to operate accurately. Depending on how you use your system, your controller will recalibrate either every 35 days or every 42 days. Your system can be used in one of the following two ways:

(a) If you turn your system off daily when it is not in use:

If you turn your emit off when it is not in use, you will notice that your system displays information every time you switch it from STANDBY to ON or from ON back to STANDBY. First, your system displays the number of days until it is going to recalibrate. The format for this is the following;
CAL XX YY
   Where “XX” is the number of days, and “YY” is the number of hours until the system is going to recalibrate. For example,
CAL 40 21 means that the system will recalibrate in 40 days and 21 hours.
   Next, your system displays the number of days until the sensor module needs to be replaced. This is discussed further below.
   (b) If your system is left on during the last 7 days before recalibration is required:

The system will automatically go into its calibration mode at the end of the 7 days. However, if you continue to turn your system from ON to STANDBY every day, your system will recalibrate when the number of days until calibrate (the “XX”) has dropped below 7 days. This feature allows you to have control over when the system will calibrate. You can then choose to switch the unit to STANDBY at a convenient time for calibration.

During calibration, your system can not be used, since your room or tent must not be at a simulated altitude. Hence, please make sure to turn your system to STANDBY at a time when you do not plan to use it for several hours.

Upon switching the emit to STANDBY with less than 7 days left until recalibration, the unit will start the recalibration process within 30 minutes.

When the calibration is complete, the number of days until the next calibration is needed is automatically reset to 42 days and 0 hours.

(c) If you leave your system ON all of the time:

If you leave your system on all the time, your Colorado Mountain Room Controller will begin to periodically display the number of days and hours until calibration during the last 7 days until recalibration. At the end of 42 days, (when the “CAL XX YY display shows CAL, 00 00) your system will automatically go into calibration mode regardless of whether you put it in STANDBY or not. During calibration, your system can not be used, since your room or tent must not be at a simulated altitude.

When the calibration is complete, the number of days until the next calibration is needed is automatically reset to 42 days and 00 hours. The unit will then resume normal operation if it is in the ON state. It will return to standby if it is switched to the STANDBY state.

During this time, you will not be able to use your system. For this reason, even if you leave your system ON all the time, it is recommended that you periodically turn your system to the STANDBY mode to see how long it is until your system needs to recalibrate, and select the time when you want it to calibrate by switching it into the STANDBY mode once you are within the last 7 days until calibration and opening the doors to the volume.

A-3.2.3.2 Manual Calibration

You can execute a manual system calibration at any time. To do this, first turn your system to STANCBY. Then, press and hold the CO2/SET POINT switch in the CO2 position WHILE you turn the system back to the ON position. Continue to hold the CO2/SET POINT switch until you see the number “0” in the left display and a multi-digit number in the right display. You may now release the CO2/SET POINT switch.

In this mode, the UP/DOWN arrows keys scroll through 2 different information readouts, 0 and 1. Press the UP/DOWN arrow key once, you should see an “1” in the left display. You may ignore the information in the right display. Again, press the CO2/SET POINT switch to the CO2 position and release. The display should now show “OPEN door”. This indicates that the calibration process has started. Soon, the displays will change from “OPEN door” to “CAL”. Switch the unit back to the STANDBY mode. It will continue to display “CAL” in the display, until the calibration process is complete. At that time, it will turn itself back off. This can take 10 minutes to as much as a couple of hours, depending on conditions.

NOTE: If you do not turn your unit back to STANDBY, it will keep showing CAL in the display until you do so.

A-3.3 Sensor Module Replacement:

The sensors in your sensor module wear out with time. Therefore, they must be replaced every year or every 365 days. This replacement interval is well within the specified life of the sensors. Every time that you put your system in the STANDBY state, your controller will display first the time to calibration, and then the number of days until you must replace the sensor module. The format for this is as follows:
o2 ZZZ

Where “ZZZ” is the number of days until your sensor module needs to be replaced. As the time to replace your sensor module approaches, your system will display the number of days until it must be replaced during normal operation. For example,
o2 317 means that the sensor module needs to be replaced in 317 days

During the final month of your sensor module, your display will read a number less than 30 days. During this month, CAT Customer Service will contact you to arrange shipment of a new sensor module to you. If you are not contacted, please contact the Colorado Altitude Training Service Department to order a replacement module. The module is:

Part Number 1359: Colorado Mountain Room Controller Sensor Module

This module is user replaceable and the instructions for installing the sensor module in your controller are included with the module.

Once the number of days until module replacement reaches zero, you have a 29 day grace period to replace the module. During this time the unit will display the days as a negative and will display the days more frequently.

After the 29 day grace period is finished, the unit will be inoperable until the sensor module is replaced.

A-3.4 Display Self-Test:

Each time that the controller is turned on, it performs a display self-test. During this test, the display momentarily shows all “8's” in the windows. This allows the user to confirm that all of the segments in the display are working properly.

A-3.5 Pressure Averaging:

The current atmospheric pressure is measured by the unit periodically. This pressure is used to calculate the actual altitude where the system is installed. Because the atmospheric pressure varies with changes in weather, it is normal for the calculated simulated altitude to change from day to day. Your controller will take a pressure reading on a regular basis over several days and average the atmospheric pressure at your elevation, thereby “learning” what altitude it is at. Hence, the variation in day to day ACTUAL, reading should decrease somewhat with time.

A-3.6 Error Message:

Your controller is continuously sensing many parameters for your safety. The controller contains dual, redundant oxygen sensors. If for any reason the outputs from these two sensors do not agree, the system will generate an alarm. If this alarm occurs, switch the unit into STANDBY for a few minutes. Then switch the unit on again and determine if the error still exists. If the error condition is removed, the unit will continue operating normally after switching to STANDBY and then to ON. If the error still exists, contact CAT customer service for assistance at 1-877-258-4883.

The following tips will help you in the daily operation of your CAT system:
1. The system should be turned at least one to two hours before use to allow the simulated altitude to stabilize. The exact amount of time that your system will require to come up to altitude will depend on the size of the tent or clear room and the altitude set point. Turn your system off and allow your room to “air out” when not in use.
2. The controller unit must be in the vertical position to function properly. This is best achieved by mounting it on a wall or stand using the keyhole opening in the back of the unit.
3. Do not block the CAT logo vent holes on the front of the unit. These holes allow sample air to reach the sensors inside the unit.
4. Do not open the controller unit. Doing so will void your warranty. The only user serviceable part is the small rectangular plate labeled “CMR Controller Sensor Module”, which is removed from the back of the unit.
5. Enter your room as quickly as possible. Doing so minimizes the loss of hypoxic air and hence, simulated altitude.
6. Clean the air filter on top of the air units at least once a month; more frequently if needed. The filter is easily removed by gently pulling it off of your air unit. Wash it gently under warn water using a mild detergent solution. Rinse the filter thoroughly and squeeze out the excess water. Allow the filter to dry thoroughly before putting it back on your air unit.
7. Remember to keep bedding and other objects away from the air units.
8. Check the air units from time to time to ensure that their flow meter continues to show flow of 10 L/min during normal operation.
9. If power is applied to the unit and the display appears to continue to be in STANDBY, verify that the brightness control is turned clockwise.

### APPENDIX B: COLORADO MOUNTAIN ROOM CONTROL SYSTEM INSTALLATION GUIDE

Setting Up Your Colorado Mountain Room Controller

1. Find a suitable location for your controller. The controller should be mounted up against one of the walls of your tent or Clear Room in a location away from the door to the enclosure. It should also be away from the location where the air from the air unit(s) enters the enclosure, and be near a power outlet. It is a good idea to mount the controller so that the bottom of the controller is not more than 50 inches above the floor. This will allow the power supply for the controller to sit on the floor rather than hang from the unit.

2. Remove the power supply and power cord from the shipping box. Connect the connector on the end of the power supply output cable to the corresponding socket on the bottom the controller, on the left side. Ensure that the power plug is the correct type for your country. Plug the power cord into the power supply. DO NOT plug the power supply into the wall at this time.
NOTE: It is recommended that you connect the controller to an Uninterruptible Power Supply. This will keep the controller functioning in the event of a power outage.

3. Find a suitable place to locate the air unit(s). There are several requirements that dictate a good location for your air unit(s). First, the location must have climate control. The climate requirements are as follows:
Temperature Range: 50-104 Degrees F.
   Humidity Range: 30-95% Relative Humidity
   Environment: Smoke, pollutant and fume free
Power Requirements: 120 VAC Units: 7 amps per unit, therefore 2 units per 20 amp circuit 230 VAC Units: 3 amps per unit

These units have a compressor and do make some noise. Place them out of the way and preferably at a distance from the bed. Placing the unit on something soft like carpet will help to mute sound and vibration. Be sure that nothing is blocking the bottom or top vent of the unit. Ensure that the power cord is correct for your country and plug the unit into the wall. It should not come on at this time.

4. Use the following table to determine what channel the air units should be connected to:

Number the units in your installation and connect them to the proper channel of “Main”, “Boost A”, or “Boost B”.

| Air Unit # | Connect to channel |
|---|---|
| 1 | M |
| 2 | A |
| 3 | M |
| 4 | A |
| 5 | B |

For example: If your installation has 2 air units, connect the first unit to “M” and the second unit to “A”. If a third is ever added, it would be connected to “M” and so on following the table.

5. After using the table to determine the proper channels for the air units, connect the RJ45 control cable(s) (provided) to the proper port on the bottom of the controller. Route the cable(s) out of the enclosure through one of the wire entry ports near the floor, and then route them in an out-of-the way manner, such as along a wall and behind furniture to the air unit(s). Connect the RJ45 control cable to either control port on the air unit. Make sure you press the cables all the way in until the locking mechanism clicks.

6. To connect an additional air unit to a control channel, daisy-chain the units together. Connect the RJ45 control cable to the air unit and to another air unit that is connected to the proper channel.

7. Scrubbers (if applicable) are also controlled via the RJ45 control cables. If equipped, locate your scrubber in an away corner of your enclosure. Route its power cord out of the enclosure through one of the wire entry ports near the floor, and plug it into a wall socket. Connect the RJ45 control cable from the scrubber to port “B' in the base of the controller. Alternatively, if you are using port B on the controller for air units, the scrubber can be plugged into any open RJ-45 socket on an air unit. In this instance, the control cable will have to be run back into the enclosure to the scrubber.

8. Connect the output of the Air Units to the enclosure using the supplied plastic hosing. Uncoil the tubing and locate the end with a gray connector on it. Plug this connector into the mating socket on the front of the air unit. Route the tubing in an out-of-the way manner, such as along a wall and behind furniture, taking care to ensure that there are no kinks in the tubing. Route the other end into your enclosure through one of the tube entry ports at the top of a wall, at the end of the enclosure where the head of the bed will be. Plug the air unit(s) into wall power. Note that they will not come on until commanded to do so by the controller.

9. At this point, installation is completed. Before plugging in the controller, refer to the Colorado Mountain Room Control System User Guide for instruction on first time operation.

[image]

[image]

[image]

### APPENDIX D: A PRACTICAL APPROACH TO ALTITUDE TRAINING by Edmund R. Burke, Ph.D. University of Colorado at Colorado Springs Colorado Springs, Colo., 80933, USA

The use of altitude training to augment sea level endurance performance is widely practiced by athletes and coaches. Over the last decade several carefully controlled studies that show that altitude training can improve sea level endurance performance, above and beyond good sea level training, when certain conditions are met. If potential pitfalls are avoided and the athlete lives high enough for long enough, they will increase erythropoientin (EPO), red cell mass, and hemoglobin. Training at low altitude while living high allows athletes to work at an intensity similar to sea level.

The “live high-train low” strategy proposes that athletes can improve sea level endurance performance by living high (2,000-3,000 meters/6,500-9,000 feet) for a minimum of three weeks and training simultaneously at a low elevation (less than 1,000 meters/3,300 feet). This “high-low” altitude training leads to the enhancement of sea level V02 max, and endurance performance.

However, the practicality of moving to attitude for extended periods of time is beyond the means and cost of many athletes due to occupational, school and family commitments. In an effort to reduce the financial and logistical challenges of traveling to altitude training sites, scientists and manufactures have developed artificial altitude environments that simulate the hypoxic conditions of moderate altitude. Endurance athletes in many sports have recently started using hypoxic tents and rooms as part of their altitude training programs. The practicality of this is seen in the ease of using these devices, portability, cost and effectiveness of long term use in releasing EPO, significantly increasing red blood cell (RBC) count and improving performance.

The altitude tent and room and the living high and training low approach provide the best approach for the enhancement of the sea-level performance in athletes. Professional and amateur athletes and Olympic Training Centers worldwide use nitrogen houses, or hypoxic rooms and tents to reach peak performance. When it comes to effectiveness, ease of use and ethical considerations they offer the athlete a fair, safe and cost effective altitude training system.

Background

Over the last few decades many athletes and coaches have used altitude training in various forms to help improve an athletes performance both for competing at altitude and at sea level. The traditional approach was to move on a permanent basis to an area, which afforded an increased altitude (1,880 to 2,000 m/5,000 to 6,000ft) and adequate terrain to allow the athlete to train in their particular sport.

Communities, such as Boulder and Colorado Springs, Colo., and Flagstaff, Ariz., became popular training and residence sites for athletes to take advantage of the hypoxic (low oxygen) environment, climate, and training terrain. However, this approach is not always feasible or affordable for most athletes to consider. Many individuals because of family, their profession, or schooling cannot take advantage of a permanent move to such training environments. Some athletes have used the concept of going to such sites for several weeks on a periodic basis during one's training cycle. This approach also leads to tremendous expense and logistical problems.

In the mid 1990's a new theory of altitude training became popular because of its scientific basis and ability of athletes to maintain quality and intensity training that is often compromised while training at altitude.

Several coaches and athletes now base their altitude training programs on the living high and training low hypothesis, whereby athletes live and recover at moderate altitude (2,500 m/8,200 ft) but train at lower altitude or sea level. The rationale behind this hypothesis is that physiological benefits are attained by living at moderate altitude, while workout volume and intensity are maintained by training at a lower altitude (below 1,250 m/4,100 ft).

Drs. Ben Levine, Jim Stray-Gundersen, Heikki Rusko and Dick Telford have conducted most of the research on the living high and training low hypothesis. Data collected by these scientists on collegiate distance runners and other athletes who completed several weeks of living high and training low training demonstrated the following results:
Faster 5-kilometer run time.
Improvement in maximal aerobic capacity.
Improvements in critical blood markers.
Lower heart rates and blood lactate levels while working at submaximal workloads. —“Altitude effect” lasted for 2-3 weeks after returning to sea level.
   Literature on the Effects of Living High, Training Low

Recently, Drs. Arnie Baker and Wil Hopkins, conducted a review of the literature on the effects of living high and training low on subsequent sea-level performance (1998). The following summarizes their findings.

One group of researchers studied athletes who lived and trained at altitude but breathed oxygen enriched air during hard training to simulate training low. Five studies involved athletes living on a mountain at 2,500 m and descending to 1,250 m on most days to train. In the other two studies, the athletes trained at sea level but got altitude exposure equivalent to 2,200-3,000 m by spending most of the time in a “nitrogen house” flushed with air containing more nitrogen and less oxygen than normal. The average athlete in almost all of these studies showed an improvement in endurance and overall performance within the first week of return from altitude, and in most studies, the improvement was definite.

The only researchers to look beyond a week after returning from altitude are Levine and Stray-Gundersen (1997), with a group of runners. Several weeks after returning from altitude, the athletes in the high-low group showed a trend towards further improvement, the average improvement relative to performance before altitude exposure is probably 2-3%.

Drs. Baker and Hopkins go on to explain that the average athlete can expect an enhancement of performance of a few percent from living high and training low, but it is now clear that some athletes get an even bigger boost, while others may get no benefit at all. Chapman et al. (1998) have analyzed these differences between athletes, using data for sub-elite runners from several previous studies as well as data from a new group of elite runners. They classified the sub-elites as non-responders (no improvement in performance of a 5000-m run 3 days after return from altitude) or high responders (better than the average improvement of 1.4%). Of 26 sub-elites who lived high and did at least their high-intensity training at a lower altitude, 31% were nonresponders and 54% were high responders. The new group of 22 elite runners, who did their high-intensity training low but otherwise lived and trained high, had a similar average improvement (1.2%) and comparable proportions of non-responders (23%) and high responders (41%). In contrast, of 13 athletes who lived and trained high, 54% were non-responders and only 23% were high responders. These data reinforce the advantage of living high and training low over the traditional high-high training. In addition, the increased number of non-responders in each group is likely to be somewhat greater in these studies than in the general population for two reasons. First, the results are based on a test performed within a few days of return from the altitude camps, when the athletes had either not re-acclimatized to the Dallas heat or had not recovered from the detraining effect of reduced training intensity caused by training at altitude. Second, the usual 1-2% variation in an athlete's performance between tests will tend to decrease the differences in proportions of responders and non-responders.

What accounts for the individual differences in the response to altitude exposure? There has always been a concern that better athletes might respond less because they might have less headroom for improvement, but that's clearly not the case here. Previous work by the Drs. Levine and Gundersen had identified inadequate iron stores as a contributing factor (Stray-Gundersen et al., 1992) to lack of adaptation to altitude. Extra iron is needed for the increase in production of red cells stimulated by exposure to altitude. But in their more recent work, all athletes had been given iron supplements to offset any iron deficiency. The authors could not identify any other blood test, lab test, or physical characteristic that would help predict which athletes were more likely to benefit from an altitude camp. There were clear differences after the camp: the high responders had a greater and more sustained increase in the concentration of erythropoietin, and they also ended up with a substantial increase in volume of red blood cells and in maximum oxygen uptake.

Drs. Rusko and Stray Gunderson have also stated that a minimum stay of 10 to 12 hours per night for a minimum of three to four weeks are required to see the benefits of living high and training low.

According to scientific research reported above and studies sponsored by the U.S. Olympic Committee, living at high altitude and training at low altitude provides improvements in speed and endurance. The reason for this is that your body adapts to altitude by increasing the blood's oxygen-carrying capacity, as well as your ability to use that oxygen. And that helps you go faster, longer and more efficiently at any elevation, from sea level to high altitude.

Physiological Effects of Altitude

From the above reported research and that of other scientists, it is obvious that living high and training low is an effective and safe method of training. The well-documented physiological effects of altitude include:
Increased natural hormone erythropoietin (EPO) production, which in turn increases red blood cell mass for delivering oxygen to muscle cells and converting it into energy.
A boost in total blood volume to move oxygen more efficiently through your bloodstream.
An increase in V02 max—the maximum amount of oxygen the body can convert to work, giving you more stamina for the long haul.
Cranked-up hematocrit levels to provide a greater percentage of cells carrying oxygen.
Elevated capillary volume, creating more blood pathways to muscle cells for improved muscle oxygenation.
A higher volume of mitochondria—the powerhouses in cells that help your body turn oxygen into energy.
An increase in the lungs' ability to exchange gases efficiently—so that every breath you take more oxygen gets into the bloodstream.

However, permanently moving to moderate altitude or taking periodic trips to altitude has logistical problems. The ability for someone to move and live in a place such as Park City, Utah, and periodically train at a lower altitude such as Salt Lake City, has drawbacks similar to the original practice of moving to altitude—a financial and logistical impact. There is also the logistical problem of having to travel back and forth from high altitude to lower altitude on a daily basis for adequate training.

The main problem is a shortage of suitable high altitude training venues, so for most athletes this option means the expense and stress of international travel and of living away from home for up to a month. Loss of heat acclimatization may also be a problem if the high and low training venues are too cool.

Artificial Altitude Environments

In an effort to reduce the financial and logistical challenges of traveling to altitude training sites, scientists and manufactures have developed artificial altitude environments that simulate the hypoxic conditions of moderate altitude.

How it Works

While you are sleeping in the thin air inside your high altitude environment, reduced quantities of oxygen diffuse across your lungs' walls into the blood. Once this modestly oxygenated blood reaches your kidneys, special kidney cells sense the lower than-normal oxygen levels and stimulate the production EPO. EPO journeys through the blood stream to the bone marrow, where it steps up the production of red blood cells. The number of red cells in your blood gradually increases, and repeated sleeps in your high-altitude bedroom eventually leave you with blood as viscous as a high altitude native's. Your blood's oxygen-carrying capacity is up, and you've become a better runner the easy way—by “training” while you sleep.

The following lists the altitude training devices and procedures being used to increase one's red blood cell mass and endurance capacity in addition to “live high, train low and training at altitude:

Nitrogen House/Room

The nitrogen house is located in Finland and was built because of that country's lack of an altitude-training site. The nitrogen house is a standard-sized living structure that simulates the reduced oxygen level conditions of 2,500-m (8,200-ft) altitude by maintaining the air inside the house at higher levels of nitrogen and lower levels of oxygen in the house. Research conducted by Finnish sport physiologist Heikki Rusko on six elite cross-country skiers suggests that training in the nitrogen house is just as effective as training at altitude. Specifically, Dr. Rusko found that changes in critical blood markers, submaximal heart rate, and submaximal. Lactate was similar among athletes who trained in the nitrogen house compared to athletes who trained at an altitude camp (Rusko, 1996).

A nitrogen house can be built almost anywhere as a fixed or mobile facility. However, it may not be very cost-effective, costs can be in the hundreds of thousands of dollars to build such houses. In addition, athletes will have to tolerate living in a dormitory environment away from home. Colorado Altitude Training (www.altitudetraining.com) manufactures a system that will convert virtually any room to an altitude room at a much lower cost. It also offers altitude tent systems at an even lower cost, making altitude training within the reach of all but the poorest athletes.

Supplemental Breathing of Oxygen During Exercise

Supplemental oxygen is used to simulate either normoxic (sea level) or hyperoxic conditions during high-intensity workouts at altitude. This method is a modification of the ‘high-low’ strategy, since athletes live in a natural terrestrial altitude environment but train at ‘sea level’ with the aid of supplemental oxygen breathe in by mask during exercise. Limited data regarding the efficacy of hyperoxic training suggests that high intensity workouts at moderate altitude (1,860 m/6,100 ft) and endurance performance at sea level may be enhanced when supplemental oxygen training is utilized at altitude over a duration of several weeks (Morris, 2000).

Certain sports such as swimming and team sports would find it impossible to train with supplemental oxygen. Breathing supplemental oxygen during exercise does not provide the benefits of altitude acclimatization.

Brief Exposures to Intermittent Hypoxic Exposure

Several devices are available that allows one to breath oxygen-depleted air through a face mask for an hour or two, several times a day. The air has an oxygen content of 10-12%, equivalent to approximately 5,000 in (17,000 ft).

Intermittent Hypoxic Exposure (IHE) is based on the assumption that brief exposures to hypoxia (1.5 to 2.0 hours) are sufficient to stimulate the release of EPO, and ultimately bring about an increase in RBC concentration. Athletes typically use IHE while at rest, or in conjunction with a training session. Data regarding the effect of IHE on hernatological indices and athletic performance are minimal and inconclusive (Rodriguez, 2000).

Use Erythropoietin (EPO) or Blood Doping.

There is no doubt that some top athletes have been taking injections of erythropoietin to get the increase in red blood cell mass that normally accompanies altitude exposure. There are no published scientific reports of its effectiveness with athletes, but non-athletes experienced an enhancement in peak running speed of 17% (Ekblom and Berglund, 1991). Intravenous infusion of extra red cells (blood doping) has a similar effect (Sawka et al., 1996).

However, both strategies are dangerous: the blood becomes so thick that there is a risk of sudden death from blood clotting. In addition, altitude exposure may be more effective anyway, if the increased buffering capacity (ability to tolerate high blood lactic acid values) of muscles that seems to occur with altitude exposure contributes to the enhancement of performance.

Lastly, The International Olympic Committee and practically all sports governing bodies bans the use of EPO.

Hypoxic Tent and Room

A version of a nitrogen house, in the form of a tent or room has recently appeared on the market. Tents are available in two versions. The practical version fits over the top of a queen or king size bed and allows for movement around the bed while creating a high altitude environment. A smaller version about the size of a one person camping tent is more portable and can be used by athletes who traveling frequently to different training sites.) However some manufacturer's tents suffer from high CO2 levels and uncomfortable humidity. The better systems include a means of filtering CO2 and controlling humidity.

One also has the ability to seal off a complete room to be used not only for sleeping by all as a place to allow one to spend additional hours during the day in a hypoxic environment working, watching television or relaxing.

The better designed tents simulate altitudes of up to 4000 m (5000 to 12,500 ft) and can be modified to simulate up to 4000 m (14,000 ft). The tent is set around a bed or on the floor. The advantages are substantial: it is truly portable; it can be used with little or no disruption of family life, study, or work; and it is easily the best way to establish the altitude and program of exposure that suits the individual.

The hypoxic tent system creates a hypoxic environment within the tent via a patented air separation unit that continually pumps low oxygen content air into the tent. Inside the tent the total pressure stays the same, and the oxygen content (%) reduces—so the partial pressure of oxygen is reduced. This allows the user to obtain the advantages of altitude training from any location. It's like having your own portable mountain. Again, the better tents have a CO2 scrubber to remove the build up of carbon dioxide being produced by metabolism.

There is also the option of adapting and sealing off a bedroom of one's house into an altitude room. This is more expensive than a tent, but affords the opportunity of having a bedroom in one's house set-up as a high altitude environment to not only sleep in, but as an area to spend additional hours during the day reading, working or watching television.

An hypoxic tent or room can be used to assist in the acclimatization process for individuals who live at or near sea level and plan to travel to higher altitude destinations. Skiers, runners, mountain bikers, and non-athletes often travel to higher altitudes and are affected by the reduced oxygen concentration at altitude. By using the system before traveling to higher altitudes, acclimatization can start weeks ahead of time. This produces a more comfortable and enjoyable trip.

Recent research has shown hypoxic tents and rooms to be an effective way to use the “sleep high, train low” model of altitude training (Shannon, 2001 and Ingham, 2001). Ingram showed a 13 percent increase in run time to exhaustion after sleeping in a hypoxic tent from 2500 to 3500 meters over a four week period. Shannon reported that athletes where able to sleep comfortably in an altitude tent simulating an altitude of 2500 meters.

The papers cited above support the use of a tent or room allowing one to easily fulfill the requirements of Drs. Rusko and Stray Gundersen that a minimum stay of 10 to 12 hours per night for a minimum of three to four weeks are required to see the benefits of living high and training low.

It is crucial both for safety and to ensure proper adaptation to altitude that one consider the quality and accuracy of the altitude control system when making such a purchase. One must consider the accuracy of the altitude controlling unit, CO2 elimination, quietness and control of humidity and temperature.

Altitude Sleeping Chamber

A hypobaric chamber that can simulate altitudes of up to 5,500 m (18,000 ft) and is designed to allow athletes to “sleep high, train low.” This device consists of a rigid cylinder little bigger than a person, with windows at each end and a vacuum pump attached. It has been available commercially for several years.

Like the nitrogen tent, it can be used at home, but it's too cramped to accommodate a partner. It's also twice the price of an altitude tent, less easy to use, and less transportable. It is also more noisy and uncomfortably warm.

The Practical Approach to Altitude Training

Endurance athletes in many sports have recently started using hypoxic tents and rooms as part of their altitude training programs. The practicality of this is seen in the ease of using these devices, portability, cost and effectiveness of long term use in releasing EPO, significantly increasing red blood cell (RBC) count and improving performance.

Traveling to altitude for training camps, particularly for athletes who are coming from sea level, creates greater than normal stress on the body due to the decreased availability of oxygen in the air. Consequently, training volume and intensity levels must be reduced. This causes a detraining effect because of a decrease in either training volume or/or intensity.

Unlike the constant hypoxic exposure to living and training in the mountains, the “intermittent” hypoxia of living/sleeping for approximately 10 hours a day gradually adapts the body to perform better not only in a low-oxygen (altitude) environment, but also substantially better in a normal oxygen, or “normoxic,” environments of sea level.

The practicality of moving to a high altitude sleeping location and traveling several times per week to a lower altitude to train is also impractical. The cost, time and logistics are beyond the means of most athletes.

Using a hypoxic sleeping devices lets you sleep high and train low wherever an athlete calls home by converting your existing bedroom into a an altitude room. The portability of these devices also allows them to be transported to a university dorm, training camp or competition.

Ethics of Altitude Training and Use of Altitude Simulators

There is some concern among coaches, athletes and the scientific community that the use of high altitude tents and rooms may be unsafe and unethical for use in sports given the concern these days of increased drug use by athletes in many sports.

International governing bodies of sports will declare a sporting practice banned if it causes injury, or it gives the athlete a technological advantage that is too expensive or too new for most other competitors to use. There HAS been discussion recently as to whether the different methods of altitude exposure are dangerous or offer a technological advantage that should be banned for use by athletes (Baker and Hopkins, 1998).

Nitrogen houses, hypoxic rooms and tents would be dangerous if the simulated altitude was high enough and long enough to raise the viscosity (thickness) of blood to an unsafe level. For example, an individual using a hypoxic tent might set the altitude too high, but so far there have been no reports for banning these devices on the grounds of health, safety or medical incidences. There have been no reports of an hematocrit of over 50 percent in athletes who have used an altitude tent or room

It also seems unlikely they will be banned as an expensive innovation, because they are no more expensive than the high-tech equipment used in training or performance by many athletes in sports such as cycling, skiing, bobsled, etc.

If they aren't unsafe, are they unethical? No, because you can't ban normal altitude training, so it's unfair to ban a safe practice that makes it easier or cheaper for athletes to achieve the same effect. There is no physiological difference between altitude in a tent or in the mountains—it is the same oxygen level. Recently, the Norwegian Olympic Committee has come forward with a position statement supporting the use of altitude houses falls within the ethical norms which sport follows (Norwegian Olympic Committee, 1998).

Recently Dr. David Martin, physiologist at Australia Institute of Sports gave a summary of his thoughts on the use of altitude training and use of altitude tents for training by athletes. He states that he and his colleagues at the Australia Institute have read many scientific studies published in reputable journals suggesting that some moderate altitude exposure protocols are beneficial for the elite athletes. The use of a simulated altitude chamber is safe, legal and potentially effective. Many of the coaches and athletes I work with would consider me unethical if I did not do everything in my power (legally of course) to ensure that they were not at a disadvantage at major competitions because they did not use altitude effectively.

Further, he points out that injecting EPO bypasses the stimulus—physiological response association and this is the problem because the stimulus—physiological response association and the genetic and environmental factors that influence this relationship is essentially what training for sport is all about.

The basic goal of training is to use a variety of external stimuli (exercise, environmental conditions, nutritional therapies, etc.) to produce a physiological adaptation. The key point is that injecting EPO bypasses the training stimulus, and the same goes for taking any other drug. Also, it is easily possible to increase athletes' EPO concentrations beyond their natural limits using an injection. However, an altitude chamber does not do this, although it does make it a lot easier for athletes to increase their EPO levels—just not beyond their natural limits.

In summary, governing bodies are unlikely to outlaw altitude simulation for 4 reasons:
Regulations are motivated by a concern for safety. When used properly hypoxic tents/rooms are completely safe and creates no ill side effects.
Altitude is a natural alternative to drugs. Many officials at Governing Bodies see altitude simulation as a godsend that improves performance without risk to the athletes' health. Altitude training may supplant the use of illegal and dangerous drugs.
Governing bodies seldom like to pass unenforceable regulations. Enforcing a ban on altitude or altitude simulation would be nearly impossible. There are no tests for altitude or altitude simulation. Unless governing bodies institute midnight raids on residences, it would be difficult to enforce a rule that essentially regulates where a person sleeps, or trains.
There are no intellectual arguments to distinguish between true altitude and altitude simulation—both work by inducing low oxygen levels in the blood, triggering the body's natural acclimatization response.
   Sleep High, Train Low and Win

Professional and amateur athletes and Olympic Training Centers worldwide use nitrogen houses, or hypoxic rooms and tents to reach peak performance. When it comes to effectiveness, ease of use and ethical considerations they offer the athlete a fair, safe and cost effective altitude training system.

In conclusion, the altitude room can be used to simulate moderate altitude living atmosphere at sea level and to stimulate EPO at sea level in athletes, and the living high and training low approach seems to give all the benefits of altitude acclimatization and seems to have the potential to avoid the problems related to normal altitude training. Finally, these new aspects—the altitude tent and room and the living high and training low approach—seem to provide the best approach for the enhancement of the sea-level performance in athletes.

### APPENDIX E: A METHOD FOR SIMULATING ALTITUDE BASED UPON INSPIRED PARTIAL PRESSURE OF OXYGEN

1 Introduction

Throughout high altitude physiology, attempts have been made to better define representative altitudes for research studies. These altitudes are often defined through the Standard Atmospheres or modifications of these atmospheres. These Standard Atmosphere models were originally developed for areas such as aerospace design. By establishing common conditions for pressure, temperature etc. it became possible to compare performance data between different design concepts. The Standard Atmosphere models are useful for comparing different systems. However, they are limited, since they define a pressure altitude and do not fully address the necessary parameters for a simulated “physiological altitude.”

Within high altitude physiology, a standardized reference for altitude is needed to allow comparisons between studies and for the development of a control algorithm for altitude simulation systems. This model should allow for a comparison of partial pressures for oxygen and define a “physiological altitude.” By utilizing an accepted model for pressure altitude, consistency can be maintained between studies and comparisons between results can be made. The model should define a significant physiological state, such as partial pressure of oxygen, and then the simulator can control to this state. This document develops one potential method for controlling a simulated altitude to using the inspired partial pressure of oxygen.

This model is developed through a reference to the pressure altitude model developed by Dr. West³. The desired altitude is correlated to a calculated inspired partial pressure of oxygen. An algorithm is proposed which controls the actual environmental conditions to the desired inspired partial pressure of oxygen. The impact of the environmental conditions of temperature, pressure and humidity on the model is also examined.

1.1. Variable Table

The following table defines the primary variables utilized in this analysis, particularly those in sections 3 and 4.

| Variable | Description | Units | Type |
|---|---|---|---|
| h | Altitude | km or feet | S |
| Nₒᵢ | Oxygen sensor counts measured | — | M |
| | at calibration | | |
| O_{C} | Perceived oxygen level in | (ppm*10⁻⁶) | C |
| | atmosphere by the sensor at | | |
| | calibration due to humidity | | |
| O_{DA} | Current value for dry air | (ppm*10⁻⁶) | C |
| | concentration of O2 | | |
| O_{di} | Normal dry air oxygen | (ppm*10⁻⁶) | S |
| | concentration (.20947) | | |
| O_{M} | Current O2 reading - corrected | (ppm*10⁻⁶) | C |
| | for P and T | | |
| O_{R} | Uncorrected current measure- | (ppm*10⁻⁶) | M |
| | ment for O2 | | |
| Pₐₜₘ | Calculated atmospheric pressure | torr | C |
| Pₐₜₘᵢ | Atmospheric pressure at | torr | M |
| | calibration | | |
| P_{atmM} | Current measured value of | torr | M |
| | atmospheric pressure | | |
| P_{H2O} | Partial pressure of water in | torr | C |
| | the atmosphere | | |
| P_{H2Oi} | Partial pressure of water in | torr | C |
| | the atmosphere at calibration | | |
| P_{H2OM} | Current value of partial | torr | C |
| | pressure of water in the | | |
| | atmosphere | | |
| P_{Oi} | Partial pressure of oxygen in | torr | C |
| | the air at calibration | | |
| P_{OM} | Current value of partial | torr | C |
| | pressure of oxygen in the air | | |
| P_{O2} | Partial pressure of oxygen in | torr | C |
| | the air | | |
| P_{RM} | Current partial pressure of | torr | C |
| | dry air following adjustment | | |
| | for humidity | | |
| Pₛₐₜ | Saturation pressure at the | torr | C |
| | current temperature | | |
| Pₛₐₜᵢ | Saturation pressure at the | torr | C |
| | point of calibration | | |
| P_{satM} | Saturation pressure for the | torr | C |
| | current measurement | | |
| P_{sat98.6} | Saturation pressure inside | torr | S |
| | the lungs | | |
| PI_{O2} | Set-point for inspired partial | torr | C |
| | pressure of oxygen | | |
| PI_{I} | Inspired partial pressure of | torr | C |
| | oxygen at calibration | | |
| PI_{M} | Current calculated value of | torr | C |
| | inspired partial pressure of | | |
| | oxygen | | |
| RH | Relative humidity (%/100) | — | M |
| RHᵢ | Relative humidity (%/100) at | — | M |
| | calibration | | |
| RH_{M} | Relative humidity (%/100) for | — | M |
| | current measurement | | |
| Tᵢ | Temperature at calibration | ° F. or ° C. | M |
| T_{M} | Temperature at calibration | ° F. or ° C. | M |

Note:
Alternate units may be used for all variables as long as the appropriate conversion factors are included.
C—Calculated, M—Measured, S—Set by software/Input by user

2. Atmospheric Model Development

Several models have been developed to relate atmospheric conditions to altitude. These include the hydrostatic equation and standard atmospheres.

2.1. Hydrostatic and Hypsometric Equations

Through the hydrostatic equation, the pressure varies with altitude by the relationship:

$$\frac{\partial P}{\partial Z} = - \frac{g}{R * T} * P \qquad ( 1 )$$

If constant temperature and acceleration due to gravity are assumed, the equation becomes

$$Z = - \frac{R * T}{g} * ln ( \frac{P}{{P}_{0}} ) \qquad {( 2 )}^{1}$$

Equation 2 is referred to as the hypsometric equation. The values in Eq. 2 are defined as:
Z=altitude above sea level in feet
R=Universal gas constant=53.35 (ft lbf)/(° R lbm)
Pₒ=Sea Level Pressure=2116.224 lbf/ft²
g=sea level acceleration due to gravity=32 ft/s²
T=sea level temperature=518.67° R
P=Pressure at altitude=lbf/ft²

Eq. (2) can be solved for the pressure as a function of altitude as

$$P = {P}_{0} * {ⅇ}^{- ( \frac{g * Z}{R * T} )} \qquad ( 3 )$$

The expression given in Eq. (3) can be used to estimate a pressure at ally given altitude.

2.2. US Standard Atmosphere

An alternate model of the atmosphere is the U.S. Standard Atmosphere 1976, which, for altitudes below 36,089 feet (11 kilometers) is:

$$P = {P}_{0} * {( 1 - \frac{Z}{145442} )}^{5.255876} \qquad ( 4 )$$

The values in Eq. (4) are defined in the same manner as those in Eq. (2).

2.3. Alternate Standard Atmosphere

Dr. West defines an alternate standard atmosphere for use in pressure approximations in Reference 3. This alternate model, utilizes the supplemental standard atmospheres defined in 1966 in reference 4. Dr. West utilizes the mean value of the models for 15° N for all months and 30° N in July. Dr. West states that the pressure at a given altitude can be calculated from the equation:
Pₐₜₘ=e^{(6.63268-0.1112*h-0.00149*h}^{²}⁾ (5)

In Eq. (5), the pressure is in Torr and the altitude, h, is in kilometers. Eq. (5) can be solved for expected altitude in terms of pressure by taking the natural log of each side to give:
0.00149*h²+0.1112h−6.6326+ln(Pₐₜₘ)=0 (6)

Utilizing the quadratic equation where:
a=0.00149,
b=0.1112, and
   c=(−6.6326+ln(Pₐₜₘ))

Eq. (6) can be solved for the altitude at a given pressure as

$$h = \frac{- .1112 \pm \sqrt{( {.1112}^{2} - 4 * 0.00149 * ( - 6.6326 + ln ( {P}_{at m} ) ) )}}{( 2 * 0.00149 )} \qquad ( 7 )$$

Eq. (7) can be simplified using the positive value for the radical to give altitudes above sea level as:
h=−37.315+{square root over ((5843.85−671.141·ln(Pₐₜₘ)))} (8)

Equations such as these are very useful when trying to determine the expected pressure at a given altitude within the assumptions of the model chosen. However, they are not as useful when attempting to determine an altitude that corresponds to a given pressure or when attempting to develop an algorithm to control an altitude simulation system for physiological effect.

An examination of Eq. (5), shows that small perturbations in tie altitude will result in even smaller perturbations in the calculated pressure. However, Eq. (8), indicates that small perturbations in pressure result in large changes in altitude. Therefore, Eq. (8) must be used cautiously when applied to a controller for simulated altitudes.

3. Definition of “Physiological Altitude”

The development of a system that is used to simulate the hypoxic affects of altitude must be controlled around parameters which have physiological significance. For a hypoxic system, a logical choice for this parameter is the inspired partial pressure of oxygen in the lungs. The inspired partial pressure of oxygen ultimately determines the ability of the body to absorb oxygen from the lungs into the blood. In normal air, the dry air percentage of oxygen is consistent at 20.948. The partial pressure of oxygen in the atmosphere at 0% relative humidity is
Pₒ₂=0.20947*Pₐₜₘ (9)

However, humidity displaces oxygen and therefore the effective partial pressure of oxygen is reduced by the equation:
Pₒ₂=0.20947*(Pₐₜₘ−P_{H20}) (10)

In Eq. (10), the partial pressure of water is calculated from the given relative humidity at the current temperature by determining the saturation pressure of the water from the steam tables and using the equation:
P_{H 20}=RH*Pₛₐᵣ (11)

Within the lungs, the air temperature is increased to body temperature and the relative humidity increases to 100 percent, (RH=1). Therefore, the inspired partial pressure of oxygen can be calculated through Eq. (10) as:
PIₒ₂=0.20947*(Pₐₜₘ−P_{sar98.6}) (12)

To define the physiological altitude, a model is needed that correlates the inspired pressure of oxygen in Eq. (12) to an altitude. This can be accomplished through the use of one of the “pressure altitude” models discussed in section 2. Through the use of Eq. (5) and converting from meters to feet, the “physiological altitude” equation from Eq. (12) is:
PIₒ₂=0.20947*(e^{(6.63268-0.1112*h/3280.8-0.00149(h}^{²}^{)/3280.8}^{²}⁾−P_{sar98.6} ) (13)

The humidity of the system or atmosphere is not normally critical to high altitude physiologists. This is because as the air is inhaled, it is heated and humidified in the lungs to body temperature and saturation. Therefore, as long as the dry air % O2 remains constant, this humidification process is constant and the atmospheric pressure is all that is needed to calculate the partial pressure of the oxygen in the lungs.

In a hypoxic tent, rather than vary the pressure, the % O2 is modified, Thus, the environmental conditions become important in controlling for hypoxia. In a hypoxic environment, such as a tent, the changes in the pressure and dry air concentration of oxygen both impact the final inspired partial pressure of oxygen. Changes in temperature and humidity must be monitored so that their impact on the measured value of % O2 can be calculated.

3.1. Graphical Representation of Physiological Altitude

The concept behind this method of simulated altitude is detailed in the following series of pictures. This representation has been simplified to allow the basic concept of altitude simulation to be clearly depicted by setting the dry air oxygen percentage to 20% and assuming a constant atmospheric temperature. FIG. 1 shows a representative volume of dry air at sea level pressure. In this model, the oxygen content of air is approximated to 20%. As the air is inihaled, it is humidified atid water molecules displace molecules of nitrogen and oxygen from the lung space until saturation is reached. This representation assumes that the partial pressure of water at saturation is equivalent to the displacement of 10 molecules within this volume. Note that the minimal impact of the temperature change from the room air to the temperature in the lungs is not included.

[image]

Next, as altitude increases, the pressure decreases resulting in a reduction in the number of molecules on an absolute basis. FIG. 2 shows the reduction of the absolute number of molecules in the representative volume due to the decrease in pressure although the relative percentage of oxygen to nitrogen has remained constant. It has been assumed that the pressure at the desired altitude is ½ the sea level pressure. The dry air composition remains the same at attitude, but the number of molecules in the volume for this example decreases to ½ the original amount. When the air is inhaled, the partial pressure of water at saturation remains the same, so that there are still 10 molecules of water displacing oxygen and nitrogen within the volume in the lung space. In this case, the number of remaining oxygen molecules drops to 16 from 18 representing an 11.1% absolute reduction in oxygen. Therefore, to simulate this altitude, the number of oxygen molecules in the sea level hypoxic environment must also drop to 16.

[image]

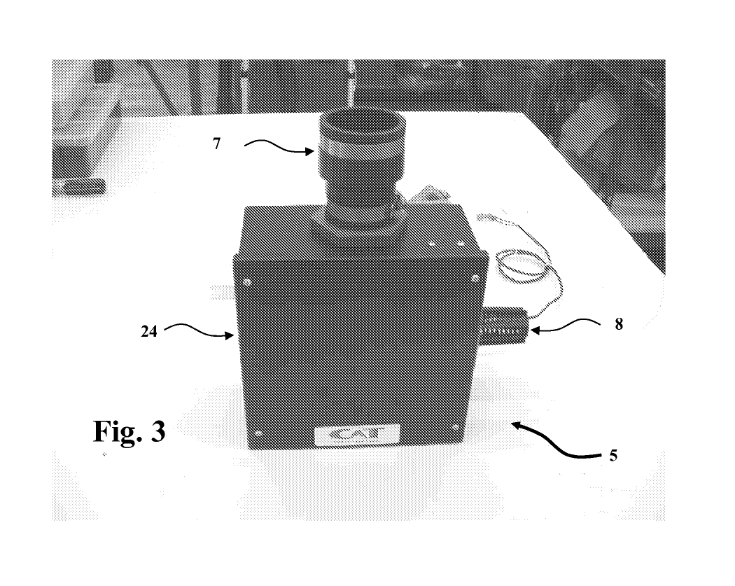

If the dry air percentage of oxygen is dropped to 10%, then the effect on the inspired oxygen is shown in FIG. 3. The oxygen is only reduced to 17 from 18 and does not completely replicate the 16 oxygen molecules of the higher altitude shown in FIG. 2.

[image]

Therefore, the dry air percentage needs to be reduced to a value less than the expected value of 10% to truly replicate the inspired partial pressure of oxygen at altitude. Thus, the control can not be set up based upon the atmospheric percentage of oxygen, but rather should be based upon the calculated inspired partial pressure of oxygen as described in section 4.

4. Simulation of “Physiological Altitude”

When simulating a physiological altitude, the correct parameters must be monitored from the point of calibration. The initial parameters to be recorded at calibration are Patmi, RHᵢ and Tᵢ. The raw reading from the oxygen sensor, Nₒᵢ, is also recorded and corrected for the errors introduced by pressure and temperature on the sensor. The reference voltage from the sensor is also recorded at this time. From these values, the saturation pressure can be determined from the steam tables as a function of temperature.
Pₛₐₜᵢ=ƒ(Tᵢ) Eq. (14)

The partial pressure of water in the atmosphere is then given by the equation:
P_{H2Oi}=RHᵢ*Pₛₐₜᵢ Eq. (15)

The partial pressure of oxygen at calibration in the atmosphere can be written through the use of Eqs. (15) and (10) as
Pₒᵢ=O_{di}*(Pₐₜₘᵢ−RHᵢ*Pₛₐₜᵢ) Eq. (16)
Where,
O_{di}=0.20947 Eq. (17)

The perceived concentration of oxygen in the atmosphere at calibration adjusted for the impact of relative humidity, i.e. what the sensor perceives as the level of oxygen, is the ratio of the partial pressure of oxygen to the measured atmospheric pressure.

$${O}_{c} = \frac{{P}_{oi}}{{P}_{at m i}} \qquad Eq . ( 18 )$$

Substituting in from Eq. (16), this expression becomes,

$${O}_{c} = {O}_{di} * ( 1 - \frac{{RH}_{i} * {P}_{sati}}{{P}_{a tmi}} ) \qquad Eq . ( 19 )$$

The value calculated in Eq. (19) is set as the reference oxygen concentration related to the measured reference voltage or initial reading, Nₒᵢ.

The inspired partial pressure of oxygen at calibration can be determined by modifying Eq. (12) with the parameters measured and calculated at calibration. This is expressed as:
PI_{I}=O_{di}*(Pₐₜₘₗ−P_{sat98.6}) Eq. (20)

During operation, readings are taken of the oxygen, atmospheric pressure, temperature and relative humidity. These values are averaged over the appropriate number of data points and the oxygen value is corrected for the impact of pressure and temperature on the sensor accuracy. Therefore, the measured oxygen concentration is given as a function of the raw data reading, temperature and pressure expressed in Eq. (21) as:
O_{M}=ƒ(O_{R},P_{atmM},T_{M}) Eq. (21)

The function in Eq. (21) is dependent on the type and brand of sensor selected for the system Eq. (14) is utilized with the current conditions to determine the current value of the saturation pressure as:
P_{satM}=ƒ(T_{M}) Eq. (22)

Therefore, the current condition for the partial pressure of the water vapor is
P_{H2OM}=RH_{M}*P_{satM} Eq. (23)

The overall ratio of oxygen in the total atmosphere as read by the sensor is given from Eq. (18) for the current conditions as:

$${O}_{M} = \frac{{P}_{oM}}{{P}_{atmM}} \qquad Eq . ( 24 )$$

The partial pressure of oxygen can be determined from Eq. (24) and the measured values of O_{M} and P_{atmM} as:
P_{OM}=O_{M}*P_{atmM} Eq. (25)

The partial pressure of the dry air is determined by subtracting the partial pressure of the water vapor from the atmospheric pressure as shown in Eq. (26).
P_{RM}=(P_{atmM}−RH_{M}*P_{satM}) Eq. (26)

The dry air concentration of oxygen can then be calculated from the partial pressures and by substituting from Eqs. (25) and (26) as

$${O}_{DA} = \frac{{P}_{oM}}{{P}_{RM}} = \frac{{O}_{M} * {P}_{atmM}}{{P}_{atmM} - {RH}_{M} * {P}_{satM}} \qquad Eq . ( 27 )$$

Finally the current level of inspired partial pressure of oxygen can be determined by utilizing Eqs. (27) and (12) with the current conditions as:

$$\begin{matrix}{PI}_{M} = {O}_{DA} * ( {P}_{atmM} - {P}_{sat 98.6} ) & Eq . ( 28 ) \\ {PI}_{M} = \frac{{O}_{M} * {P}_{atmM}}{{P}_{atmM} - {RH}_{M} * {P}_{satM}} * ( {P}_{atmM} - {P}_{sat 98.6} ) & Eq . ( 29 )\end{matrix}$$

4.1. Conversion Back to Altitude

In some cases, the display of a simulated altitude is preferred to a display of the inspired partial pressure of Oxygen. To convert back to an altitude, Eq. (8) can be modified using Eq. (20) with the current value of inspired partial pressure of oxygen to give:

$$h = [ \begin{matrix}- 37.315 + \\ \sqrt{( 5843.85 - 671.141 * ln ( \frac{ {PI}_{ M}}{ {O}_{ di}} + {P}_{ sat 98.6} ) )}\end{matrix} ] * 3280.8 \qquad Eq . ( 30 )$$

In Eq. (30), the altitude has been converted to feet and the pressures are in torr.

4.2. Impact of Variations on Readings

Variations in the key parameters of temperature, humidity and pressure will cause fluctuations in the level of inspired partial pressure of oxygen. These are beyond the impact on accuracy due to temperature and pressure dependencies in the sensors. For the following examples, the system is assumed to have been calibrated with the conditions:
Tᵢ=45° F.
Pₐₜₘᵢ=1010 mbar
RHᵢ=35% RH
O_{di}=209470 ppm

The current conditions are set at
T_{M}=55° F.
P_{atmM}=990 mbar
RH_{M}=60% RH
O_{M}=various in steps of 500

A comparison of the proposed method of calculating altitude from inspired partial pressure of oxygen to the current control algorithm is shown in FIG. 4.

[image]

The two altitudes are different by almost 700 feet in some areas. This example is only one of multiple scenarios for the variation of environmental conditions from calibration. Depending on the conditions, using the current algorithm may result in altitudes which may be too high or too low.

The following sections examine the impact of errors in the readings for temperature, pressure and humidity on the calculated altitude.

4.2.1. Temperature

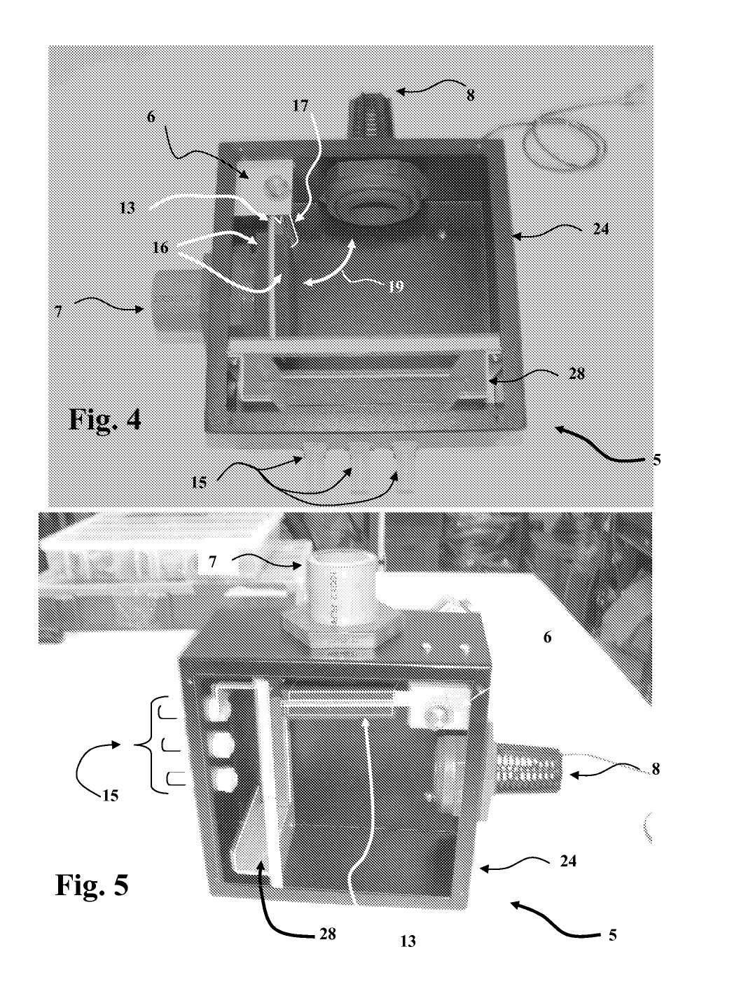

In FIG. 5, the impact of a 1% error in the temperature reading is illustrated. In this case, the measured value for temperature is set at 54.45 instead of the actual of 55 used in section 4.2. The result is a minimal impact on the calculated altitude. This shows that the temperature impact on the reading is secondary in nature. A change in the temperature changes the saturation pressure of the water vapor and therefore changes the corrected value for the oxygen concentration and the inspired partial pressure of oxygen. So, while the temperature should be regularly monitored, the measurement does not need to be as accurate as other parameters.

[image]

4.2.2. Humidity

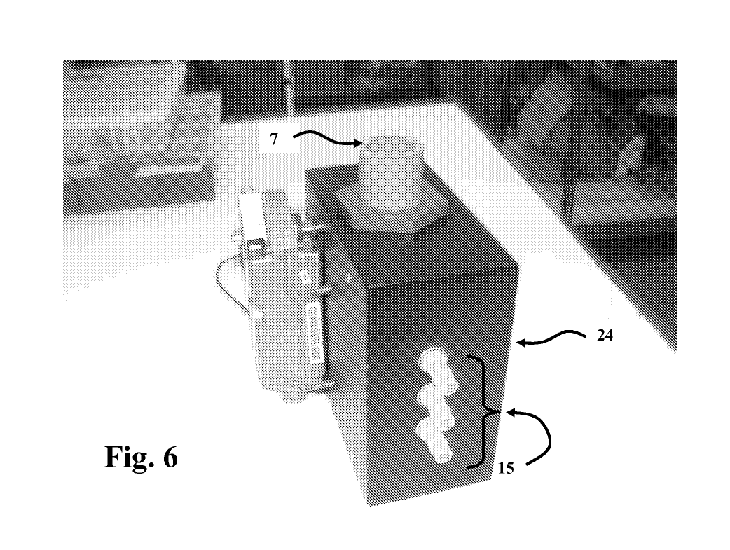

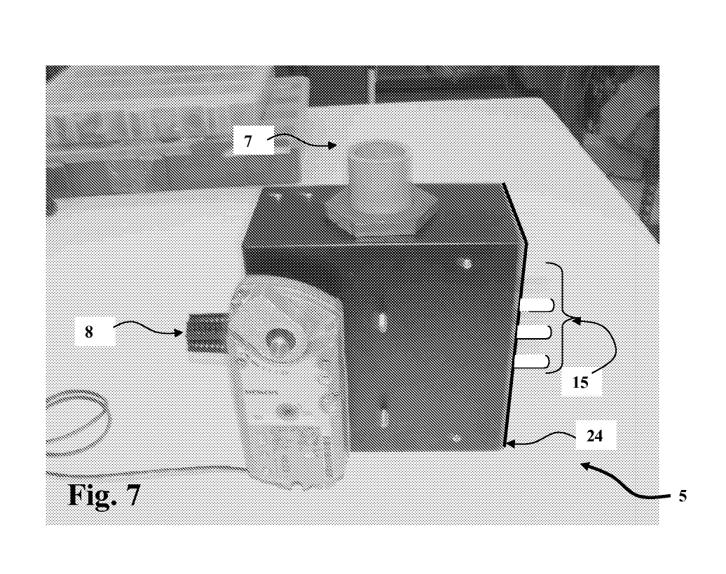

In FIG. 6, the impact from a 1% error in the humidity reading is shown. The actual humidity is set to 60% RH and the as measured value is set to 59.4% RH. Like the temperature, the relative humidity is important to establishing the proper values, but is secondary to the measurements of pressure and oxygen.

[image]

4.2.3. Pressure

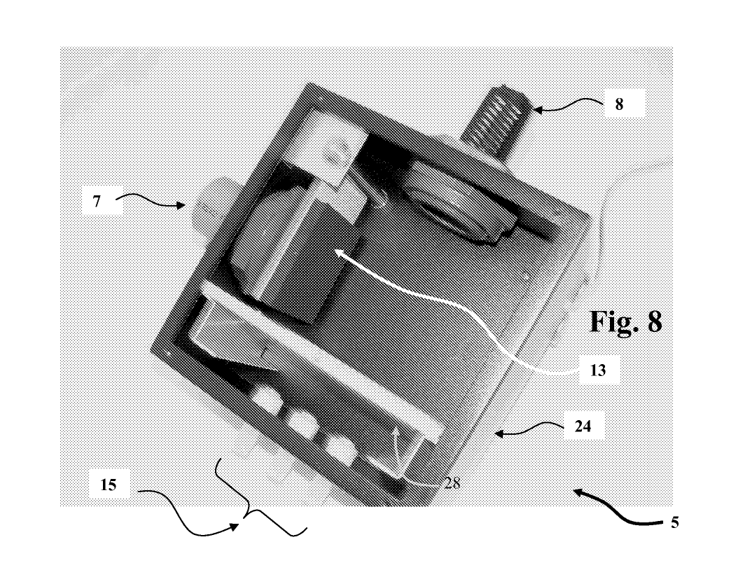

The pressure measurement has a more direct impact on the error in the calculated altitude. FIG. 7 shows the affect of a 1% error in the pressure measurement. This shift of 1% from an actual of 990 mbar to 981 mbar results in an altitude error of up to 295 feet for this situation.

[image]

As discussed in the section 2.3, any error in pressure is amplified when used to calculate an altitude through the modified West equation. The impact of this amplification of the error is clearly shown in FIG. 4. If the control algorithm is based upon the partial pressure of inspired oxygen the error from the pressure sensor is not amplified. This error is depicted in FIG. 8.

[image]

5. Simplified Algorithm

Within the controller, the desired inspired partial pressure of oxygen will be determined from the set-point altitude using Eq. (13). The parameters of pressure, humidity, temperature and % O2 are then monitored to establish the actual level of inspired partial pressure of oxygen within the environment utilizing the series of calculations listed in section 4. A simplified flow diagram of this process is shown in FIG. 9.

[image]

In addition to controlling for the altitude, the system must also monitor any parameters which could affect safety. These parameters include the levels of carbon dioxide.

### APPENDIX F: THEORY OF OPERATIONS OF THE CIRCUITRY PURPOSEFUL DELAY IN ACTIVATING AIR UNITS 3

1. SCOPE: This document shall describe the operation of the Air Unit Sequencer Controller which is used use to sequentially activate the air units 3 (FIG. 1).

2. FUNCTION: The Air Unit Sequencer Controller supplies 2.88 watts of 24 volt power required to energize the 2 relays that currently control the power on/off and oxygen valves installed in each hypoxic air generator 3. The Air Unit Sequencer Controller sequentially sends this control signal to up to 24 air units 3 to prevent line voltage sag/surge due to multiple air units 3 turning on simultaneously. It also prevents any air units 3 from being turned back on within a period of less than 30 seconds after having being turned off so that the back pressure on the compressor can be relieved prior to reactivation. This prevents local overloading of mains power lines. The Air Unit Sequencer Controller is powered from an external “brick” +24 volt power universal input supply.

3. Theory of Operation:

3.1. Power Supply: The power supply used for the Air Unit Sequencer Controller has the following features:
4000 VAC Isolation from input to output
1500 VAC Isolation from Input to ground
500 VAC isolation from output to ground
300 μA leakage max @ 264 VAC 50 Hz
3.3 A output @ 24 VDC
EN-60601-x certified
EN-55022 Class B emission certified.

Removable IEC-320 cord allows for customization for country of installation. The 24 volt return of the power supply connects to the frame of the sequencer enclosure through a spark gap and a high value resistor that allows the 24 volt return to be connected directly to a hypoxic controller 2 in another room with a possible potential difference in ground. This prevents ground faults from igniting an RJ45 cable from over dissipation.

3.2. Power Input circuitry: This section of the power supply circuit will filter the incoming +24 VDC and down regulate it to the lower voltages required for the timing and control circuits. There will be an 8 pin DIN plug on the back of the Air Unit Sequencer Controller for power input.

3.3. Sequence Cycle & Delay Logic: This section of the circuitry includes two timer circuits to provide the 500 mS and greater than 30 second intervals required by the Air Unit Sequencer Controller. When the Air Unit Sequencer Controller is powered on, OR there is a de-assertion of the input that is designated to turn the generators 3 on, the second timer for delaying at least 30 seconds is enabled. During the counting of the second times, the air unit 3 turn on output signals are inhibited. At the end of this time out, the input for a 20 bit shift register is enabled if the input for air unit 3 turn on is in the enabled state. If not, then the input to the shift register will be disabled. If the input is enabled, then the 500 mS timer will oscillate and on each rising edge, a 20 bit shift register is shifted every 500 mS until all 20 bits are in an asserted state. Each one of these bits shall be associated with the control of an output driver which, in turn, will be turned on by it. If at any time before, during, or after this shift operation the air unit turn on signal is deasserted, the shift register is cleared thereby turning off all air unit drivers and restarting the second timer. The 24 volts for this task is derived from the local power supply described in Section 3.1
The incoming O₂ valve command is driven to all respective 20 output pins any time that the input signal is true, and de-asserted any time it is false. The 24 volts for this task will be derived from the local power supply described in Section 3.1.
An additional three signals, scrub, vent, and re-circulate will be connected from the input cable directly through to the output plugs. The 24 volts for this task will be passed on from the hypoxic controller 2 upstream.

3.4. Power Output Drivers: There may be 40 total drivers for the 20 air unit on signals and the 24 for the O₂ valve control signals. The power output drivers are high side type with an automatic resetting over current protection. Since each relay load is 60 mA, this protection may be less than 1000 mA. It is not intended to connect more than two air units 3 per output line. Other wise the benefits of sequencing may be lost. The sequencer still will only be able to support 24 air units total.

3.5. 24 Output Sockets: These will be 20 standard, unshielded 8 pin RJ45 connectors with a ferrite block filter. These may be ganged or discrete.

### APPENDIX G: REFERENCES

The following references are fully incorporated herein by reference:

Allan G. Hahn, A G. and Gore, C. J. The Effect of Altitude on Cycling Performance Sports Medicine. 31(7): 533-557, 2001.

Baker, A. and Hopkins, W. G. (1998). Live-high train-low altitude training for sea-level competition In: Sportscience Training & Technology. Internet Society for Sport Science. http://sportsci.org/traintech/altitude/wgh.html

Chick, T. W., Stark, D. M., and Murata, G. H. Hyperoxic training increases work capacity after maximal training at moderate altitude. Chest. 104, 1759-1762, 1993.

Ekblom, B., & Berglund, B. Effect of erythropoietin administration on maximal aerobic power. Scandinavian Journal of Medicine and Science in Sports. 1, 88-93, 1991.

Ingham, E. A., Pfitzinger, P. D, et. al. Running performance following intermittent altitude exposure simulated with nitrogen tents. Medicine and Science in Sports and Exercise. 33(5): S2, 2001.

Levine, B. D., Stray-Gundersen, J., Duhaime, G., et al. “Living high—training low”: the effect of altitude acclimatization/normoxic training in trained runners. Medicine and Science in Sports and Exercise. 23, S25 (Abstract 145), 1991.

Levine, B. D., and Stray-Gundersen, J. “Living high-training low”: effect of moderate-altitude acclimatization with low-altitude training on performance. Journal of Applied Physiology. 83, 102-112, 1997.

Matttila, V., & Rusko, H. Effect of living high and training low on sea level performance in cyclists. Medicine and Science in Sports and Exercise. 28, S156 (Abstract 928), 1996.

Morris, D. M, Kearney, J. T. and Burke, E. The Effects of breathing supplemental oxygen during altitude training on cycling performance. Journal of Science and Medicine. 3(2): 165-175, 2000.

Norwegian Ministry of Sport Steering Committee. Press release. May. 1998

Nummela, A., Jouste, P., and Rusko, H. Effect of living high and training low on sea level anaerobic performance in runners. Medicine and Science in Sports and Exercise. 28, S124 (Abstract 740), 1996.

Nummela, A and Rusko, H. Acclimatization to altitude and normoxic training improve 400-m running performance at sea level. Journal of Sports Sciences. 18, 411-419, 2000.

Rodriguez F A, Ventura J L, Casas M, et al. Erythropoietin acute reaction and hernatological adaptations to short, intermittent hypobaric hypoxia. European Journal of Applied Physiology. 82: 170-177, 2000.

Rusko, H. R. New aspects of altitude training. The American Journal of Sports Medicine. 24(6): S48-S52, 1996.

Shannon, M. P., Wilber, R., and Kearney, J. T. Normobaric-hypoxia: Performance characteristics of simulated altitude tents. Medicine and Science in Sports and Exercise. 33(5): S60, 2001.

Stray-Gundersen, J., and Levine, B. D. Altitude acclimatization/normoxic training (High/Low) improves sea level endurance immediately on descent from altitude. Medicine and Science in Sports and Exercise. 26, S64 (Abstract 360), 1994.

Stray-Gundersen, J., and Levine, B. D. “Living high-training high and low” is equivalent to “living high-training low” for sea-level performance. Medicine and Science in Sports and Exercise. 29, S136 (Abstract 783) 1997.

Sawka, M. N., Joyner, M. J., Miles, D. S., et al. The use of blood doping as an ergogenic aid. Medicine and Science in Sports and Exercise, 28(6), i-viii, 1996 Wilber, R. Current trends in altitude training. Sports Medicine. 31(4): 249-265, 2001.

What is claimed is:

1. An air intake module for providing air to one or more hypoxic air generators, comprising:
a housing for enclosing an air passageway, wherein the passageway supplies input air to one or more hypoxic air generators that provide hypoxic air to an enclosure;
a re-circulated air intake for providing air from the enclosure to the passageway;
a fresh air intake for providing, to the passageway, air from a source exterior to the enclosure;
an air shut-off that is movable between at least: (1) a first position for allowing air from the fresh air intake to enter the passageway, and preventing air from the re-circulated air intake to enter the passageway, and (2) a second position for allowing air from the re-circulated air intake e to enter the passageway, and preventing air from the fresh air intake to enter the passageway;
an actuator for moving the air shut-off between the first position and the second position, wherein the actuator receives control information from a controller for controlling the oxygen content of the enclosure, wherein for a first range in oxygen content within the enclosure the controller outputs first control information to the actuator for providing the shut-off in the first position, and for a second range in oxygen content within the enclosure the controller outputs second control information to the actuator for providing the shut-off in the second position.

2. A hypoxic air delivery system and controller for use with patients who suffer from hypoplastic left heart syndrome comprising an oxygen concentrator, or air separation unit, a controller.

3. A hypoxic air delivery system and controller for use with patients who benefit from hypoxic preconditioning comprising an oxygen concentrator, or air separation unit, a controller.

4. A hypoxic air delivery system to provide rehabilitation to cardiac patients.

5. A means of changing oxygen content of air provided to a user comprising a pulse oximeter or other device that measures blood oxygen levels that sends a signal to a controller that modulates or turns on or off air units, such air units having the ability to provide normoxic air (approximately 21% oxygen), and/or hypoxic air (<21% oxygen) and/or hyperoxic air (>21% oxygen). normoxic air.

6. A hypoxic air delivery system and controller, comprising:
one or more hypoxic air generators; and
a corresponding manifold for each of the generators, wherein the corresponding manifold is attached its generator for containing inside the manifold, at least on component of the generator that are external to a housing of the generator, the at least one component used by the generator for generating hypoxic air.

7. A controller for an altitude simulation apparatus, comprising:
dual oxygen sensors;
a carbon dioxide sensor;
a means for delaying the activation of one or more hypoxic air generators, wherein the delaying means: (a) sequentially delays an activation of each of a plurality of the one or more hypoxic air generators, or (b) prevents re-activation of one of the generators until a predetermined elapsed time has occurred.
